(19) United States
(12) Patent Application Publication
(10) Pub. No.: US 2025/0171409 A1
(43) Pub. Date: May 29, 2025

(54) PROCESS OF PREPARATION OF INDOXACARB AND ITS INTERMEDIATES

(71) Applicant: ADAMA MAKHTESHIM LTD., Beer Sheva (IL)

(72) Inventors: Eran FOGLER, Meitar (IL); Regev PARNES, Kibbutz Mishmar Hanegev (IL); Sanaa MUSA, Deir El-Assad (IL); Natalia STRIZHIVER, Lehavim (IL); Chen VAZANA, Ramat-Gan (IL); Avi MONHAIT, Ramat-Gan (IL); Hagit FORCKOSH, Beer Sheva (IL)

(73) Assignee: ADAMA MAKHTESHIM LTD., Beer Sheva (IL)

(21) Appl. No.: 18/724,825

(22) PCT Filed: Dec. 29, 2022
(86) PCT No.: PCT/IL2022/051409

§ 371 Date: Jun. 27, 2024

Related U.S. Application Data
(60) Provisional application No. 63/296,171, filed on Jan. 4, 2022.

(51) Int. Cl. C07D 273/04; B01J 31/02; C07C 249/16; C07C 251/84; C07C 251/88; C07D 273/06

(52) U.S. Cl. CPC C07D 273/04; B01J 31/0244; C07C 249/16; C07C 251/84; C07C 251/88; C07D 273/06; C07C 2602/08

(57) ABSTRACT

The present invention relates to novel di-imine, oxadiazepine and oxadiazine intermediates as key intermediates for preparation of oxadiazine insecticides, the processes of their preparation, purification and use in the preparation of final oxadiazine pesticides, in particular of indoxacarb. Telescopic process of preparation of arthropocidal oxadiazines, in particular indoxacarb is disclosed.

### FIELD OF INVENTION

The present invention refers to new intermediates for the preparation of arthropocidal oxadiazines, novel derivative of oxadiazepine and the corresponding processes therefor. Telescopic process of preparation of arthropocidal oxadiazines, in particular, indoxacarb is disclosed.

### BACKGROUND

Arthropocidal oxadiazines and methods of their preparation along with certain key intermediates are widely disclosed for example in WO 92/11249, WO 93/19045, WO 1995/029171, WO1995/018116 and WO 1998/005656. However, preparative methods for these compounds must be improved for economic commercial operation. In view of that, the present invention makes available a convenient cost effective route to preferred arthropocidal oxadiazines.

### SUMMARY

The present invention relates to a di-imine of formula I which is racemic or enantiomerically enriched at chiral center*:

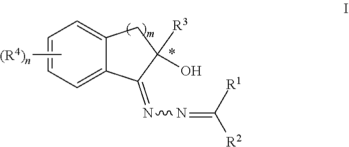

Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3

In particular, the present invention provides di-imine of formula Ia, which is racemic or enantiomerically enriched at chiral center*:

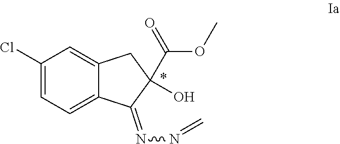 [SMILES: C=NN=C1c2ccc(Cl)cc2CC1(O)C(=O)OC]

In addition, the present invention provides a process for preparation of a di-imine of formula I, which is racemic or enantiomerically enriched at chiral center*, comprising reaction of compound of formula II

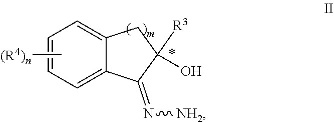

Wherein R³ is C₁-C₄-alkoxycarbonyl, COOH
R⁴ is halogen, C₁-C₄ alkyl, C₁-C₄ alkoxy
m=1-3
n=1-3
with compound of formula III or/and compound of formula IIIa

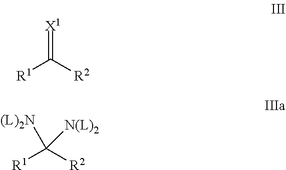

Wherein X¹=O, ⁺NL₂X⁻, L is hydrogen, C₁-C₄ alkyl, X is halogen, R¹, R² are independently hydrogen, C₁-C₄-alkyl, in the presence of organic solvent and optionally, in the presence of catalyst.

The present invention further provides a process for preparation of compound of formula Ia, which is racemic or enantiomerically enriched at chiral center*

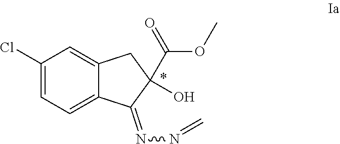 [SMILES: C=NN=C1c2ccc(Cl)cc2CC1(O)C(=O)OC]

comprising the reaction of compound of formula IIa

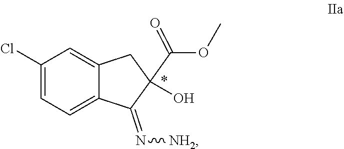 [SMILES: COC(=O)C1(O)Cc2cc(Cl)ccc2C1=NN]

with formaldehyde, and/or with N,N-Dimethylmethyleneiminium chloride (CAS 30354-18-8) and/or with tetramethylmethanediamine in the presence of organic solvent selected from the group consisting of toluene, xylene, monochlorobenzene, dichlorobenzene, ethylbenzene, cyclopentylmethyl ether, methyl-tert-butyl ether, benzonitrile and mixtures thereof, optionally in the presence of ammonium salt.

The present invention also provides a compound of formula V which is racemic or enantiomerically enriched at chiral center*:

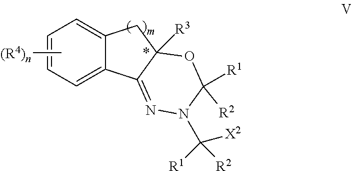

Wherein R¹, R² is independently hydrogen, C₁-C₄-alkyl
R³ is C₁-C₄-alkoxycarbonyl, COOH
R⁴ is halogen, C₁-C₄ alkyl, C₁-C₄alkoxy
X²=OR¹, N(R′)₂
m=1-3
n=1-3

In particular, the present invention provides a compound of formula Va which is racemic or enantiomerically enriched at chiral center*:

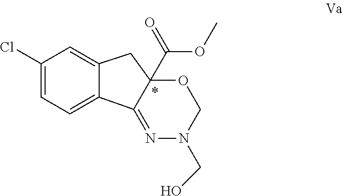 [SMILES: COC(=O)C12Cc3cc(Cl)ccc3C1=NN(CO)CO2]

In addition, the present invention provides a process for preparation of compound of formula V which is racemic or enantiomerically enriched at chiral center*,

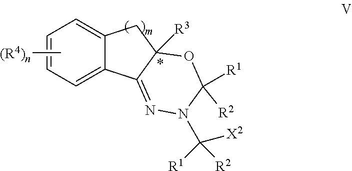

Wherein R¹, R² is independently hydrogen, C₁-C₄-alkyl
R³ is C₁-C₄-alkoxycarbonyl, COOH
R⁴ is halogen, C₁-C₄ alkyl, C₁-C₄ alkoxy
X²=OR¹, N(R′)₂
m=1-3
n=1-3
comprising reaction of compound of formula I

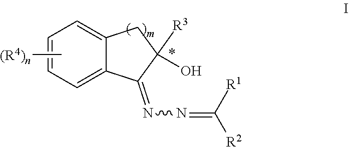

Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3
with compound of formula III or/and compound of formula IIIa

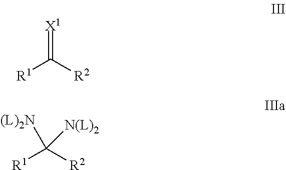

Wherein X¹=O, ⁺NL₂X⁻, L is hydrogen, C₁-C₄ alkyl, X is halogen, R¹, R² are independently hydrogen, C₁-C₄ alkyl, in the presence of organic solvent and, optionally, in the presence of catalyst.

The present invention further provides a process of preparing of compound of formula Va which is racemic or enantiomerically enriched at chiral center,

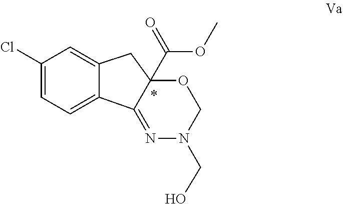 [SMILES: COC(=O)C12Cc3cc(Cl)ccc3C1=NN(CO)CO2]

the process comprising the reaction of compound of formula Ia

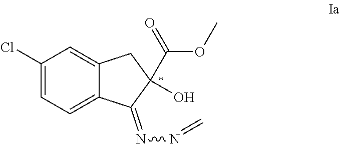

with formaldehyde in the presence of organic solvent, and optionally in the presence of catalyst.

The present invention further provides a compound of formula VI which is racemic or enantiomerically enriched at chiral center*:

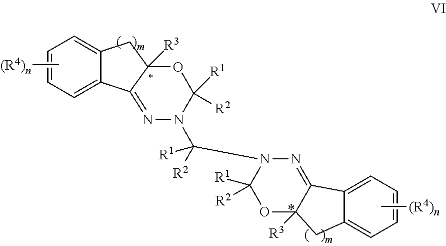

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy
m=1-3
n=1-3

In particular, the present invention provides a compound of formula VIa which is racemic or enantiomerically enriched at chiral center*:

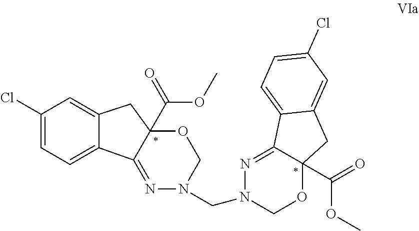

The present invention further provides a process of preparing of compound of formula VI which is racemic or enantiomerically enriched at chiral center*

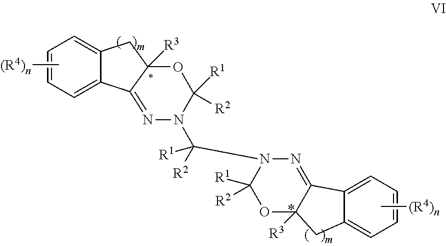

the process comprising the reaction of compound of formula I

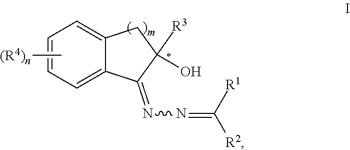

with a compound of formula V

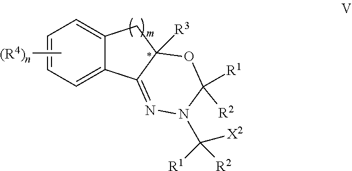

Wherein R¹, R² is independently hydrogen, C₁-C₄-alkyl
R³ is C₁-C₄-alkoxycarbonyl, COOH
R⁴ is halogen, C₁-C₄ alkyl, C₁-C₄ alkoxy
X²=OR¹, N(R¹)₂
m=1-3
n=1-3
in the presence of organic solvent and optionally, in the presence of catalyst.

In addition, the present invention provides a process of preparing of compound of formula VI which is racemic or enantiomerically enriched at chiral center*

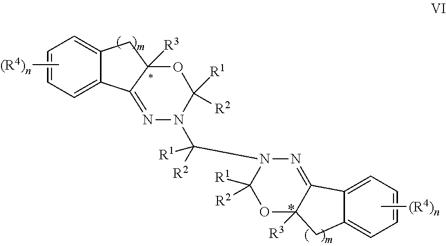

Wherein R¹, R²=hydrogen, C₁-C₄-alkyl
R³=C₁-C₄-alkoxycarbonyl, COOH
R⁴=Halogen, C₁-C₄alkyl, C₁-C₄ alkoxy
m=1-3
n=1-3
the process comprising the reaction of compound of formula V with compound of formula II

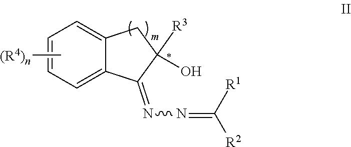

Optionally, in the presence of compound of formula III or IIIa

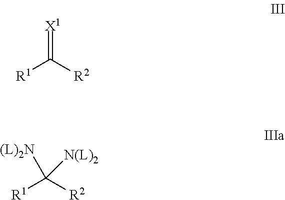

Wherein X¹=O, ⁺NL₂X⁻, L is hydrogen, C₁-C₄ alkyl, X is halogen, R¹, R² is independently hydrogen, C₁-C₄-alkyl,
in the presence of organic solvent and optionally, in the presence of catalyst.

The present invention further provides a process of preparing of compound of formula VIa which is racemic or enantiomerically enriched at chiral center*

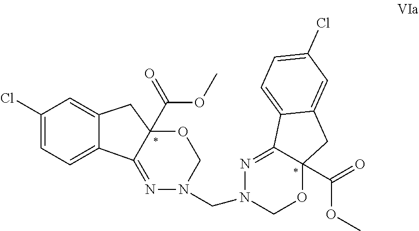

the process comprising the reaction of compound of formula IIa

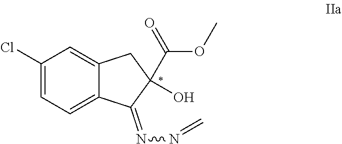

with dimethylmethyleneammonium chloride in the presence of organic solvent and optionally, in the presence of catalyst.

The present invention further provides a process of preparing of compound of formula VIa which is racemic or enantiomerically enriched at chiral center*

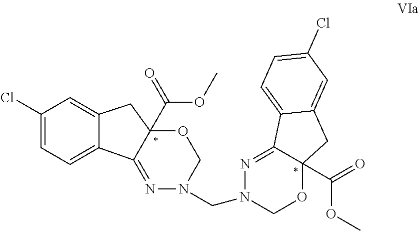

the process comprising the reaction of compound of formula IIa

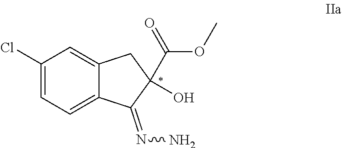

with tetramethylmethanediamine in the presence of organic solvent and optionally in the presence of catalyst.

The present invention further provides a process of preparing of compound of formula VIa which is racemic or enantiomerically enriched at chiral center*

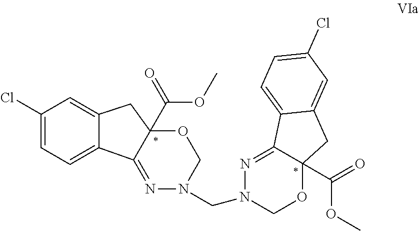

the process comprising the reaction of compound of formula Ia

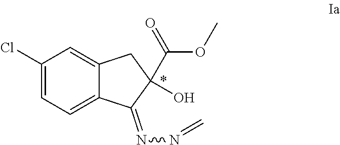 [SMILES: C=NN=C1c2ccc(Cl)cc2CC1(O)C(=O)OC]

with formaldehyde optionally in the presence of organic solvent selected from the group consisting of toluene, xylene, monochlorobenzene, dichlorobenzene, ethylbenzene, cyclopentylmethyl ether, methyl-tert-butyl ether, benzonitrile and mixtures thereof.

The present invention further provides a process for preparing of compound of formula IV which is racemic or enantiomerically enriched at chiral center*

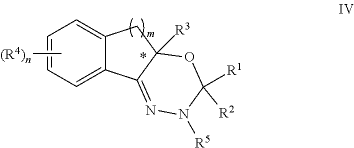

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy

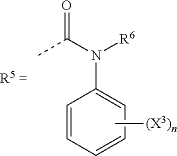

R⁶=hydrogen, CO₂Me, CO₂(R¹)
X³=Halogen, OCF₃, SCF₃
n=1-3;
the process comprising the reaction of compound of formula I

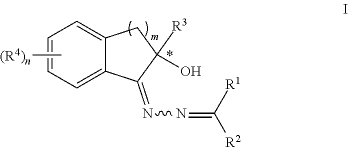

with a compound of formula VII:

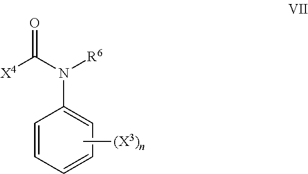

Wherein R⁶=hydrogen, CO₂Me, CO₂(R¹)
n=1-3
X³=Halogen, OCF₃, SCF₃
X⁴=Halogen, triflate, tosylate
in the presence of organic solvent.

The present invention further provides a process for preparation of compound of formula IV which is racemic or enantiomerically enriched at chiral center*

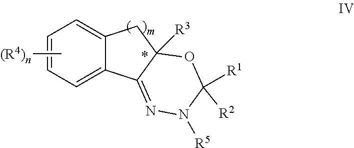

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy
R⁵=

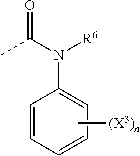

R⁶=hydrogen, CO₂Me, CO₂(R¹)
X³=Halogen, OCF₃, SCF₃
n=1-3;
comprising the reaction of a mixture containing at list one of the compounds of formula I, V and/or VI:

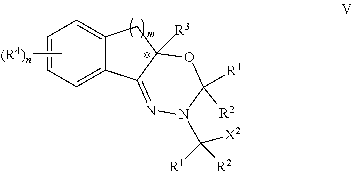

Wherein R¹, R² is independently hydrogen, C₁-C₄-alkyl
R³ is C₁-C₄-alkoxycarbonyl, COOH
R⁴ is halogen, C₁-C₄ alkyl, C₁-C₄alkoxy
X²=OR¹, N(R¹)₂
m=1-3
n=1-3

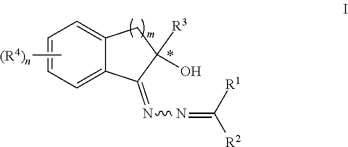

Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3

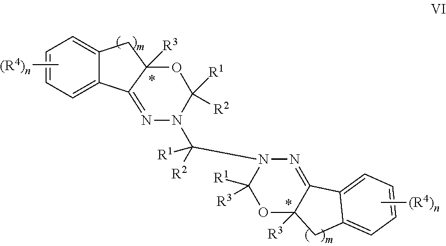

Wherein R¹, R²=hydrogen, C₁-C₄-alkyl
R³=C₁-C₄-alkoxycarbonyl, COOH
R⁴=Halogen, C₁-C₄alkyl, C₁-C₄ alkoxy
m=1-3
n=1-3
with a compound of formula VII in the presence of organic solvent.

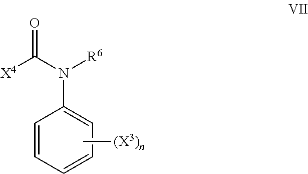

Wherein R⁶=hydrogen, CO₂Me, CO₂(R¹)
n=1-3
X³=Halogen, OCF₃, SCF₃
X⁴=Halogen, triflate, tosylate

The present invention further provides a process for preparation of compound of formula IVa which is racemic or enantiomerically enriched at chiral center*

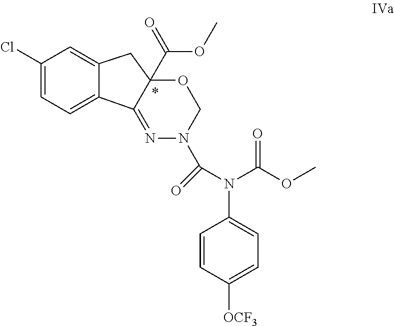 [SMILES: COC(=O)N(C(=O)N1COC2(C(=O)OC)Cc3cc(Cl)ccc3C2=N1)c1ccc(OC(F)(F)F)cc1]

comprising the reaction of compound of formula Ia

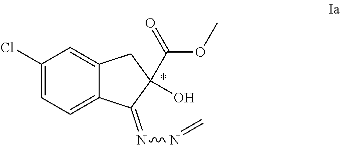 [SMILES: C=NN=C1c2ccc(Cl)cc2CC1(O)C(=O)OC]

with a compound of formula VIIa

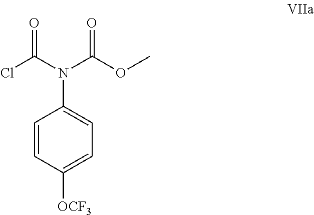 [SMILES: COC(=O)N(C(=O)Cl)c1ccc(OC(F)(F)F)cc1]

in the presence of organic solvent.

The present invention further provides a process for preparing of compound of formula IVa which is racemic or enantiomerically enriched at chiral center*

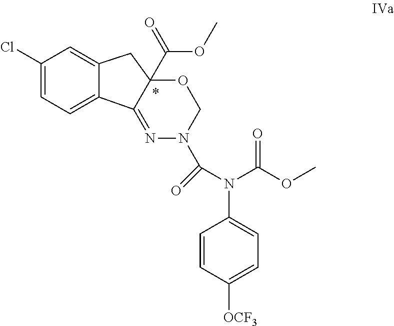 [SMILES: COC(=O)N(C(=O)N1COC2(C(=O)OC)Cc3cc(Cl)ccc3C2=N1)c1ccc(OC(F)(F)F)cc1]

the process comprising the reaction of compound of formula VIa

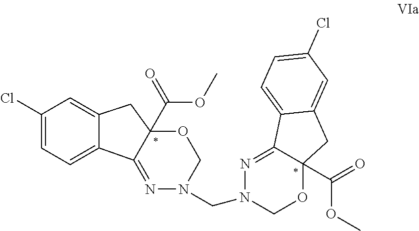

with a compound of formula VIIa

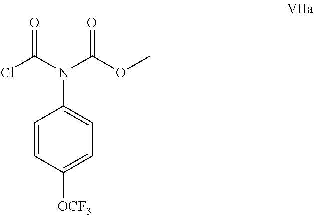 [SMILES: COC(=O)N(C(=O)Cl)c1ccc(OC(F)(F)F)cc1]

in the presence of organic solvent.

The present invention further provides a process for preparing of compound of formula IVa which is racemic or enantiomerically enriched at chiral center*

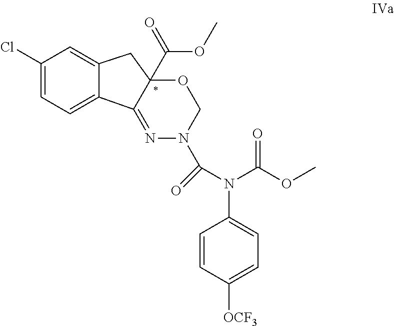

the process comprising the reaction of compound of formula Va

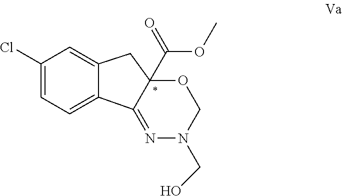

with a compound of formula VIIa

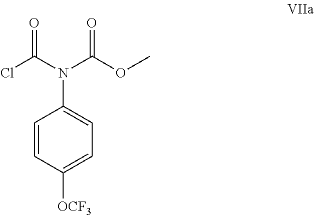 [SMILES: COC(=O)N(C(=O)Cl)c1ccc(OC(F)(F)F)cc1]

in the presence of organic solvent.

The present invention further provides a process for preparation of compound of formula IVa which is racemic or enantiomerically enriched at chiral center*

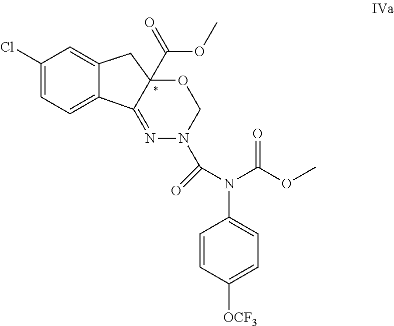

comprising the reaction of a mixture containing at list one of the compounds of formula Ia, Va and/or Via

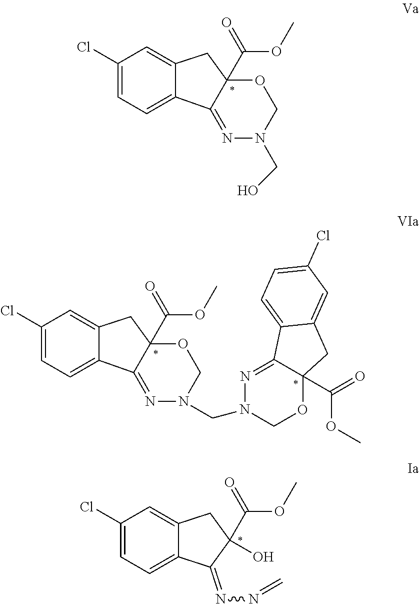

with a compound of formula Vila

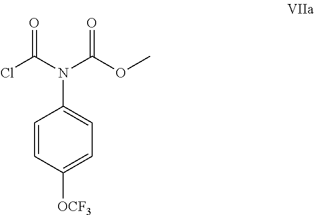 [SMILES: COC(=O)N(C(=O)Cl)c1ccc(OC(F)(F)F)cc1]

in the presence of organic solvent.

The present invention provides a telescopic process of for preparation of compound of formula IV which is racemic or enantiomerically enriched at chiral center*

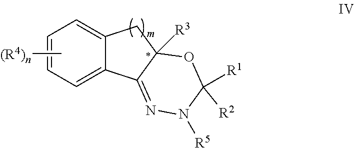

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy

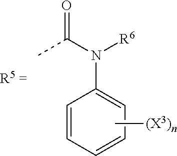

R⁶=hydrogen, CO₂Me, CO₂(R′)
X³=Halogen, OCF₃, SCF₃
n=1-3;
comprising the steps of: a) preparation of compound, or V, or VI or the mixture thereof b) reaction of compound I, or V, or VI or the mixture thereof with compound of formula VII in the presence of organic solvent.

The present invention further relates to purification of compound of formula IV by recrystallization from C₁-C₄ alcohol, preferably from isopropanol, optionally in the presence of water.

The present invention further provides a compound of formula VIII (methyl 8-chloro-2,6-dihydro-3H-indeno[1,2-e][1,3,4]oxadiazepine-5a(5H)-carboxylate which is racemic or enantiomerically enriched at chiral center*:

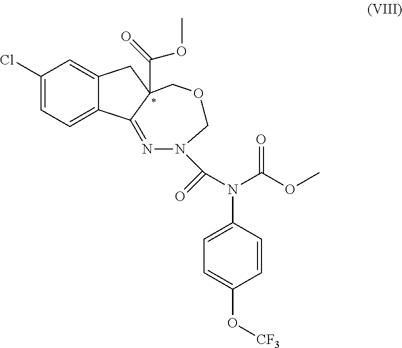 [SMILES: COC(=O)N(C(=O)N1COCC2(C(=O)OC)Cc3cc(Cl)ccc3C2=N1)c1ccc(OC(F)(F)F)cc1]

The present invention further provides a process for preparing of compound of formula VIII which is racemic or enantiomerically enriched at chiral center* according to the following scheme, comprising: a) reacting the mixture of methyl (S)-5-chloro-2-hydroxy-1-oxo-2,3-dihydro-1H-indene-2-carboxylate and (1H-Indene-2-carboxylic acid, 5-chloro-2,3-dihydro-1-oxo-, methyl ester) with hydrazine hydrate in the presence of methanol and acetic acid, b) reacting the mixture of methyl (S)-5-chloro-1-hydrazineylidene-2-hydroxy-2,3-dihydro-1H-indene-2-carboxylate and methyl 5-chloro-1-hydrazineylidene-2,3-dihydro-1H-indene-2-carboxylate prepared in a) with formaldehyde in the presence of pyridine hydrochloride, c) reacting the mixture of methyl (S)-5-chloro-2-hydroxy-1-(methylenehydrazineylidene)-2,3-dihydro-1H-indene-2-carboxylate and methyl 8-chloro-2,6-dihydro-3H-indeno[1,2-e][1,3,4]oxadiazepine-5a(5H)-carboxylate, prepared in step b) with methyl N-carbonochloridoyl-N-[4-(trifluoromethoxy)phenyl]carbamate:

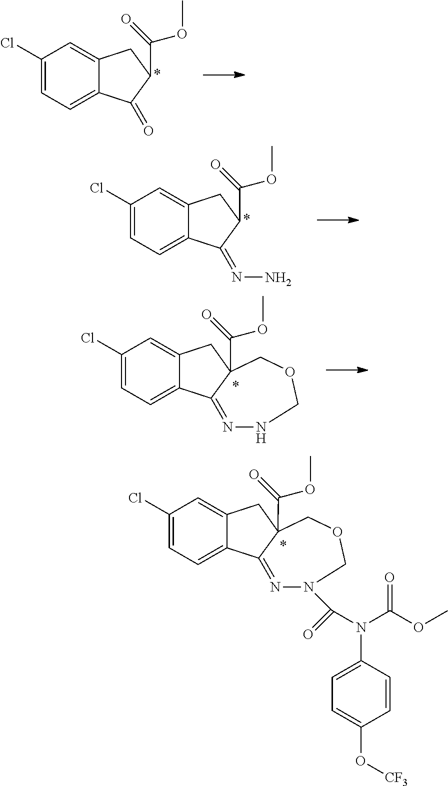

The present invention further relates to purification of compound of formula VIII by recrystallization from the mixture of isopropanol/water, wherein the ratio of isopropanol to water is 3:1.

### DESCRIPTION

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

Throughout the application, descriptions of various embodiments use the term “comprising”; however, it will be understood by one skilled in the art, that in some specific instances, an embodiment can alternatively be described using the language “consisting essentially of” or “consisting of”.

The term “a” or “an” as used herein includes the singular and the plural, unless specifically stated otherwise. Therefore, the terms “a,” “an” or “at least one” can be used interchangeably in this application.

The term “alkyl” as used herein refers to a branched, unbranched, or cyclic carbon chain. The term “halogen” or “halo” as used herein refers to one or more halogen atoms, defined as F, Cl, Br, and I. Unless indicated to the contrary, the numerical parameters set forth in the following specification and attached claims are approximations that may vary depending upon the desired properties sought to be obtained. For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.”

At the very least, each numerical parameter should at least be construed in light of the number of reported significant digits and by applying ordinary rounding techniques. In an embodiment, use of the term “about” herein specifically includes ±10% from the indicated values in the range. In addition, the endpoints of all ranges directed to the same component or property herein are inclusive of the endpoints, are independently combinable, and include all intermediate points and ranges.

The term “carbonyl” as used herein refers to the group —C═O

The term “alkoxy,” as used herein, refers to an alkyl group attached to the parent molecular moiety through an oxygen atom.

The term “alkoxycarbonyl,” as used herein, refers to an alkoxy group attached to the parent molecular moiety through a carbonyl group.

The term “alkyl,” as used herein, refers to a group derived from a straight or branched chain saturated hydrocarbon containing from one to four carbon atoms.

Certain compounds of this invention can exist as one or more stereoisomers. The various stereoisomers include enantiomers, diastereomers, and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

Accordingly, the compounds of the invention may be present as a mixture of stereoisomers, individual stereoisomers, or as an optically active form.

The term “telescopic process” as used herein refers to carrying out several reactions without isolating the intermediate products. In particular, the telescopic process suggests the execution of multiple transformations (including reaction quenches and other workup operations) without the direct isolation of intermediates. Telescoped solutions of intermediates can be extracted, filtered (as long as the desired product remains in the filtrate), and solvent exchanged, but the intermediate is ultimately held in solution and carried forward to the subsequent transformation.

The term “racemic” as used herein refers to a bulk sample of the compound containing a mixture of equal amounts of left- and right-handed enantiomers of a said compound.

The term “enantiomerically enriched” as used herein refers to a bulk sample of the compound containing an excess of either the (+) or (−) enantiomer and includes any combination greater than a 1:1(racemic) mixture of enantiomers up to and including 100% of the pure enantiomer. Thus, for example, an enriched compound having 25% (−) enantiomer and 75% (+) enantiomer is viewed as a mixture of 50% racemate and 50% pure (+) enantiomer and is referred to as having 50% enantiomeric excess of the (+) enantiomer.

Any of the compounds described here as basic compound or intermediate in a process is intended also to include the compound salts e.g HCl salts, acetic acid salts etc., no special meaning should be given to the fact that in some cases this is mention or not mention for specific compound in the text.

In a first aspect, the present invention provides a di-imine of formula I which is racemic or enantiomerically enriched at chiral center*

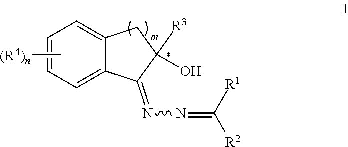

Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3

In a preferred embodiment the present invention provides a di-imine of formula Ia, which is racemic or enantiomerically enriched at chiral center, wherein R¹ is H, R² is H, R³ is CO₂Me, R⁴ is Cl, n is 1, m is 1:

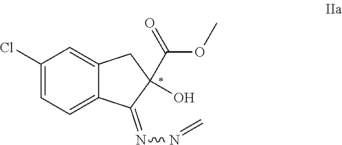

According to the present invention a compound of formula I is prepared by reaction of compound of formula II

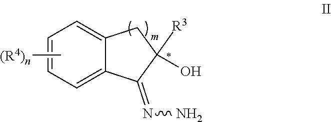

Wherein R³ is C₁-C₄-alkoxycarbonyl, COOH
R⁴ is halogen, C₁-C₄ alkyl, C₁-C₄ alkoxy
m=1-3
n=1-3
with compound of formula III or compound of formula IIIa

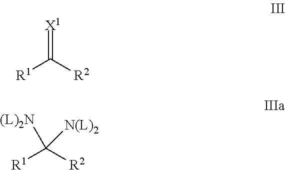

Wherein X¹=O, ⁺NL₂X⁻, L is hydrogen, C₁-C₄ alkyl, X is halogen, R¹, R² are independently hydrogen, C₁-C₄-alkyl, in the presence of organic solvent and optionally, in the presence of catalyst.

Compound of formula III is known and commercially available or could be prepared by common methods as disclosed in [Knoll, Fritz; Krumm, Ulrich Chemische Berichte (1971), 104(1), 31-9 and references whereof. Compound of formula IIIa is known and commercially available or could be prepared by common methods as disclosed in Gaudry, Michel; Jasor, Yves; Khac, Trung Bui, Organic Synthesis (1980), 59, 153-9.

According to an embodiment, the molar ratio of compound formula II to compound of formula III or IIIa is from about 3:1 to about 1:10, preferably from about 1:2 to 1:4.

According to an embodiment, the molar ratio of compound formula II to compound of formula III or IIIa is from about 1:1 to about 1:10, preferably from about 1:2 to 1:3.

According to an embodiment the aforementioned reaction of preparation of compound of formula I comprises an aprotic organic solvent.

In a further embodiment the organic aprotic solvent selected from the group consisting of toluene, xylene, monochlorobenzene, dichlorobenzene, ethylbenzene, cyclopentylmethyl ether, methyl-tert-butyl ether, benzonitrile and the mixtures thereof.

In a further embodiment the organic aprotic solvent is monochlorobenzene.

According to another embodiment the aforementioned reaction is employed in the presence of catalyst. The suitable catalyst is ammonium salt selected from the group consisting of pyridinium hydrochloride, pyridinium acetate, pyridinium triflate, pyridinium hydrobromide, tetrabutylammonium bromide, tetrabutylammonium chloride, tetrabutylammonium hydrogen sulfate, tetrabutylammonium iodide, tetraethylammonium bromide, tetraethylammonium chloride, tetraethylammonium hydrogen sulfate, tetraethylammonium iodide, tetramethylammonium bromide, tetramethylammonium chloride, tetramethylammonium hydrogen sulfate, tetramethylammonium iodide, tetrapropylammonium bromide, tetrapropylammonium chloride, tetrapropylammonium hydrogen sulfate, tetrapropylammonium iodide, tetraoctylammonium bromide, tetraoctylammonium chloride, tetraoctylammonium hydrogen sulfate, tetraoctylammonium iodide and the mixtures thereof.

Suitable amount of the ammonium salt catalyst in the process of preparation of compound I from compound II is from 0.1 to 50 mol %, preferably from 1.0 to 10.0 mol %.

Finally, the reaction mixture containing the resulting compound of formula I is worked up. This stage may include filtration, purification, pH adjustment, extraction, distilled out some or all the solvent, concentration and recrystallization.

According to the present invention, the compound of formula II preferably contacted with the compound of formula III or/and compound of formula IIIa at raised temperature. A preferred temperature interval is from −20° C. to 150° C., the most preferred interval is from 5 to 30° C.

According to another embodiment the compound of formula Ia which is racemic or enantiomerically enriched at chiral center*

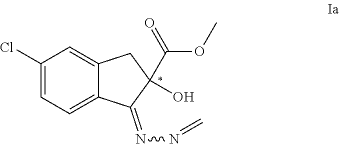

Is prepared by the reaction of compound of formula IIa

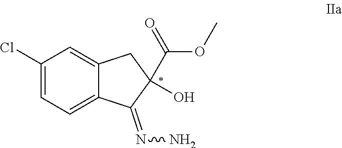

with formaldehyde, and/or with N,N-Dimethylmethyleneiminium chloride (CAS 30354-18-8) and/or with tetramethylmethanediamine in the presence of organic solvent selected from the group consisting of toluene, xylene, monochlorobenzene, dichlorobenzene, ethylbenzene, cyclopentylmethyl ether, methyl-tert-butyl ether, benzonitrile and mixtures thereof, optionally in the presence of ammonium salt. The compound of formula IIa is known and could be prepared by reacting methyl 5-chloro-2,3-dihydro-2-hydroxy-1-oxo-1H-indene-2-carboxylate with hydrazine monohydrate according to the first part of step B of Example 2 in WO 92/11249.

In a further embodiment the organic aprotic solvent is monochlorobenzene.

According to another embodiment the suitable ammonium salt is selected from pyridinium hydrochloride, pyridinium hydrobromide, tetrabutylammonium bromide and the mixtures thereof.

According to an embodiment, the molar ratio of compound formula IIa to formaldehyde, and/or to N,N-Dimethylmethyleneiminium chloride (CAS 30354-18-8) and/or to tetramethylmethanediamine is from about 5:1 to about 1:100, preferably from about 1:2 to 1:10.

According to an embodiment, the molar ratio of compound formula IIa to formaldehyde, and/or to N,N-Dimethylmethyleneiminium chloride (CAS 30354-18-8) and/or to tetramethylmethanediamine is from about 1:1 to about 1:5, preferably from about 1:2 to 1:4.

Suitable amount of the ammonium salt catalyst in the process of preparation of compound Ia from compound IIa is from 0.1 to 50 mol %, preferably from 1.0 to 10.0 mol %.

At the end of the process, the reaction mixture containing the resulting compound of formula Ia is worked up. This stage may include filtration, purification, pH adjustment, extraction, distilled out some or all the solvent, concentration and recrystallization According to the present invention, the compound of formula IIa preferably contacted with formaldehyde, and/or with N,N-Dimethylmethyleneiminium chloride (CAS 30354-18-8) and/or with tetramethylmethanediamine at. A preferred temperature interval is from −20° C. to 50° C., the most preferred interval is from 8° C. to 30° C.

Another aspect of the present invention provides a compound of formula V which is racemic or enantiomerically enriched at chiral center:

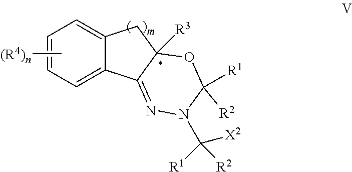

Wherein R¹, R² is independently hydrogen, C₁-C₄-alkyl
R³ is C₁-C₄-alkoxycarbonyl, COOH
R⁴ is halogen, C₁-C₄ alkyl, C₁-C₄alkoxy
X²=OR¹, N(R¹)₂
m=1-3
n=1-3

A preferred embodiment provides a compound of formula Va which is racemic or enantiomerically enriched at chiral center wherein R¹ is H, R² is H, R³ is CO₂Me, R⁴ is Cl, n is 1, m is 1, X² is OH:

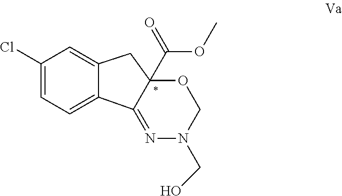

According to another aspect of the present invention a compound of Formula V which is racemic or enantiomerically enriched at chiral center is prepared by reaction of compound of formula I

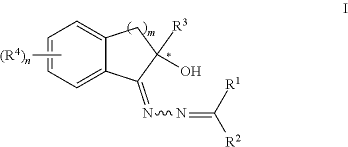

Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3
with compound of formula III or compound of formula IIIa

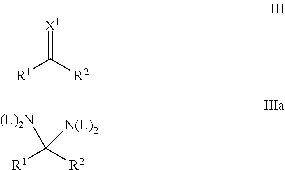

Wherein X¹=O, ⁺NL₂X⁻, L is hydrogen, C₁-C₄ alkyl, X is halogen, R¹, R² are independently hydrogen, C₁-C₄ alkyl, in the presence of organic solvent and, optionally, in the presence of catalyst.

In an embodiment the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

Preferably, the process of preparation of compound V performed in the presence of ammonium salt catalyst, which is selected from pyridinium hydrochloride, pyridinium hydrobromide, tetrabutylammonium bromide and the mixtures thereof.

According to an embodiment, the molar ratio of compound formula I to compound of formula III or IIIa is from about 1:1 to about 1:100, preferably from about 1:1 to 1:10.

Suitable amount of the catalyst in the process of preparation of compound V from compound I is from 0.1 to 50 mol %, preferably from 1.0 to 10.0 mol %.

Finally, the reaction mixture containing the resulting compound of formula V is worked up. This stage may include filtration, purification, pH adjustment, extraction, concentration and recrystallization.

According to the present invention, the compound of formula I preferably contacted with the compound of formula III or IIIa at raised temperature, i.e. over room temperature (over 20° C.). A preferred temperature interval is from 20 to 130° C., the most preferred interval is from 25 to 45° C.

According to specific embodiment of the present invention a compound of formula Va which is racemic or enantiomerically enriched at chiral center*, is prepared by the process

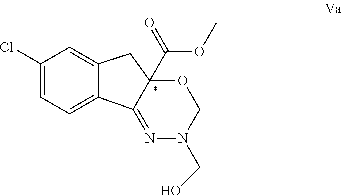

comprising the reaction of compound of formula Ia

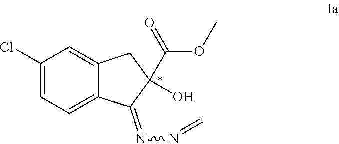

with formaldehyde in the presence of organic solvent, and optionally in the presence of catalyst.

In an embodiment the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

Preferably, the process of preparation of compound Va performed in the presence of ammonium salt catalyst, which is selected from pyridinium hydrochloride, pyridinium hydrobromide, tetrabutylammonium bromide and the mixtures thereof.

According to an embodiment, the molar ratio of compound formula Ia to formaldehyde is from about 0.6:1 to about 1:100, preferably from 1:1 to 1:10.

Suitable amount of the catalyst in the process of preparation of compound Va from compound Ia is from 0.1 to 50 mol %, preferably from 1.0 to 10.0 mol %.

Finally, the reaction mixture containing the resulting compound of formula Va is worked up. This stage may include filtration, purification, pH adjustment, extraction, concentration and recrystallization.

According to the present invention, the compound of formula Ia preferably contacted with the compound of formula III or IIIa at raised temperature, i.e. over room temperature (20° C.). A preferred temperature interval is from 20 to 130° C., the most preferred being 25 to 35° C.

Another aspect of the present invention represents a compound of formula VI which is racemic or enantiomerically enriched at chiral center*:

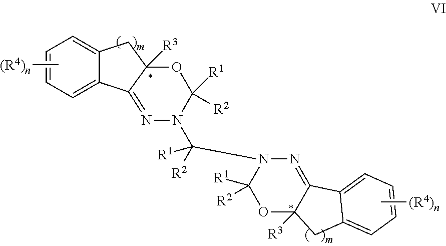

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy
m=1-3
n=1-3

According to a preferred embodiment of the present invention in the compound of formula VI which is racemic or enantiomerically enriched at chiral center, R¹ is H, R² is H, R³ is CO₂Me, R⁴ is Cl, n is 1, m is 1 as represented by formula VIa:

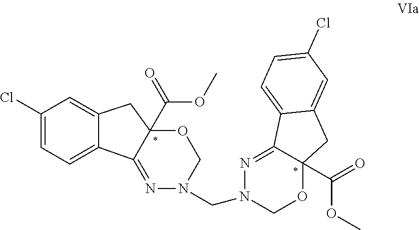

In an embodiment the present invention provides a process of preparing of compound of formula VI which is racemic or enantiomerically enriched at chiral center*

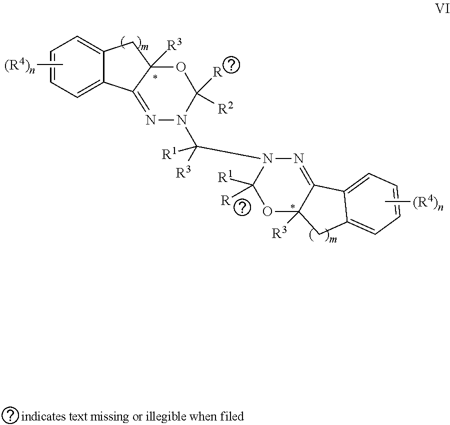

the process comprising the reaction of compound of formula I

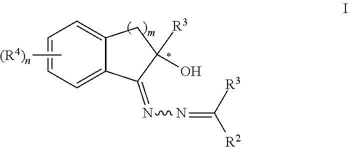

with a compound of formula V

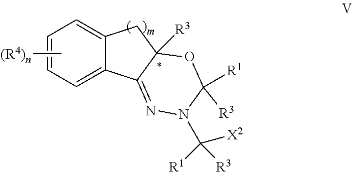

Wherein R¹, R² is independently hydrogen, C₁-C₄-alkyl
R³ is C₁-C₄-alkoxycarbonyl, COOH
R⁴ is halogen, C₁-C₄ alkyl, C₁-C₄ alkoxy
X²=OR¹, N(R¹)₂
m=1-3
n=1-3
in the presence of organic solvent and optionally, in the presence of catalyst.

In an embodiment the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

Preferably, the process of preparation of compound VI which is racemic or enantiomerically enriched at chiral center is performed in the presence of ammonium salt catalyst, which is selected from pyridinium hydrochloride, pyridinium hydrobromide, tetrabutylammonium bromide and the mixtures thereof.

According to an embodiment, the molar ratio of compound formula I to compound of formula V is from 10:1 to 1:10, preferably from 4:1 to 1:1.

Suitable amount of the catalyst in the process of preparation of compound VI from compound I is from 0.1 to 50 mol %, preferably from 1.0 to 10.0 mol %.

Finally, the reaction mixture containing the resulting compound of formula VI is worked up. This stage may include filtration, purification, extraction, concentration and recrystallization.

According to the present invention, the compound of formula I preferably contacted with the compound of formula V at temperature interval from 0 to 180° C., preferably from 25 to 50° C.

In an embodiment the present invention provides a process of preparing of compound of formula VI, which is racemic or enantiomerically enriched at chiral center *

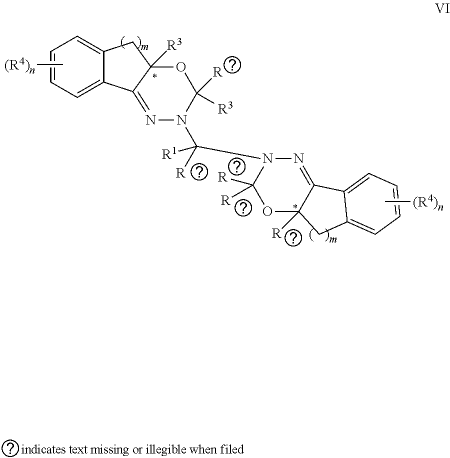

Wherein R¹, R²=hydrogen, C₁-C₄-alkyl
R³=C₁-C₄-alkoxycarbonyl, COOH
R⁴=Halogen, C₁-C₄alkyl, C₁-C₄ alkoxy
m=1-3
n=1-3
comprising the reaction of compound of formula II

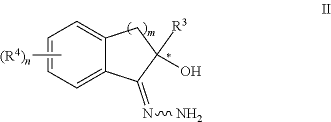

with compound of formula III or IIIa

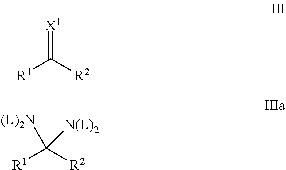

Wherein X¹=O, ⁺NL₂X⁻, L is hydrogen, C₁-C₄ alkyl, X is halogen, R¹, R² is independently hydrogen, C₁-C₄-alkyl,
in the presence of organic solvent and optionally, in the presence of catalyst.

In an embodiment the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

Preferably, the process of preparation of compound VI performed in the presence of ammonium salt catalyst, which is selected from pyridinium hydrochloride, pyridinium hydrobromide, tetrabutylammonium bromide and the mixtures thereof.

According to an embodiment, the molar ratio of compound formula II to compound of formula III or/and IIIa is from 1:1.5 to 1:10, preferably from 1:2 to 1:5.

Suitable amount of the catalyst in the process of preparation of compound VI from compound II is from 0.1 to 50 mol %, preferably from 1.0 to 10.0 mol %.

Finally, the reaction mixture containing the resulting compound of formula VI is worked up. This stage may include water addition, phase separation, filtration, purification, extraction, concentration and recrystallization.

According to the present invention, the compound of formula II preferably contacted with the compound of formula III or/and IIIa at temperature interval from 0 to 180° C., preferably from 10 to 45° C.

In a preferred embodiment the present invention provides a process of preparing of compound of formula VIa, which is racemic or enantiomerically enriched at chiral center*

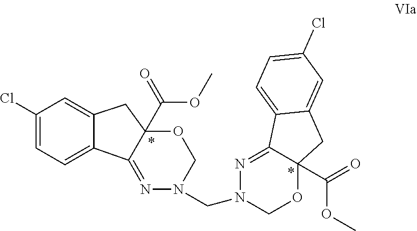 [SMILES: COC(=O)C12Cc3cc(Cl)ccc3C1=NN(CN1COC3(C(=O)OC)Cc4cc(Cl)ccc4C3=N1)CO2]

comprising the reaction of compound of formula IIa

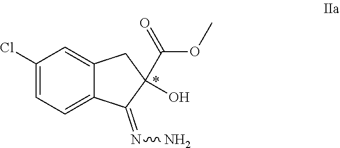 [SMILES: COC(=O)C1(O)Cc2cc(Cl)ccc2C1=NN]

with diethylmethyleneammonium chloride in the presence of organic solvent and optionally, in the presence of catalyst.

In an embodiment the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

According to an embodiment, the molar ratio of compound formula IIa to diethylmethyleneammonium chloride is from 1:1.5 to 1:100, preferably from 1:2 to 1:20.

Suitable amount of the catalyst in the process of preparation of compound VIa from compound IIa is from 0.1 to 50 mol %, preferably from 1.0 to 10.0 mol %.

Finally, the reaction mixture containing the resulting compound of formula VIa is worked up. This stage may include water addition, phase separation, filtration, purification, extraction, concentration and recrystallization.

According to the present invention, the compound of formula Ia preferably contacted with the compound of formula III or/and IIIa at temperature interval from 0 to 180° C., preferably from 5 to 55° C.

In a further embodiment the present invention provides a process of preparing of compound of formula VIa, which is racemic or enantiomerically enriched at chiral center*

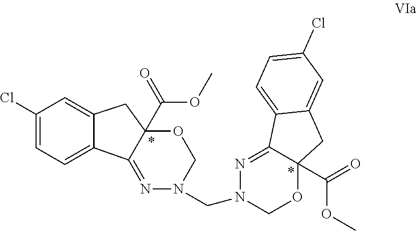 [SMILES: COC(=O)C12Cc3cc(Cl)ccc3C1=NN(CN1COC3(C(=O)OC)Cc4cc(Cl)ccc4C3=N1)CO2]

the process comprising the reaction of compound of formula IIa

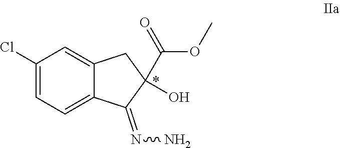 [SMILES: COC(=O)C1(O)Cc2cc(Cl)ccc2C1=NN]

with tetramethylmethanediamine in the presence of organic solvent and optionally in the presence of catalyst.

In an embodiment the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

Suitably, the process of preparation of compound VIa performed in the presence of ammonium salt catalyst, which is selected from pyridinium hydrochloride, pyridinium hydrobromide, tetrabutylammonium bromide and the mixtures thereof.

According to an embodiment, the molar ratio of compound formula Ia to tetramethylmethanediamine is from about 1:1.5 to about 1:10.0, preferably from about 1:2 to 1:8.

Suitable amount of the catalyst in the process of preparation of compound VIa from compound IIa is from 0.1 to 50 mol %, preferably from 1.0 to 10.0 mol %.

Finally, the reaction mixture containing the resulting compound of formula VIa is worked up. This stage may include water addition, phase separation, filtration, purification, extraction, concentration and recrystallization.

According to the present invention, the compound of formula IIa preferably contacted with tetramethylmethanediamine at temperature interval from 0 to 180° C., preferably from 10 to 60° C.

In a further embodiment the present invention provides a process of preparing of compound of formula VIa which is racemic or enantiomerically enriched at chiral center*

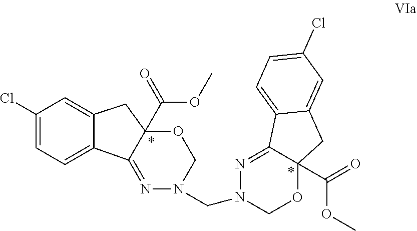 [SMILES: COC(=O)C12Cc3cc(Cl)ccc3C1=NN(CN1COC3(C(=O)OC)Cc4cc(Cl)ccc4C3=N1)CO2]

comprising the reaction of compound of formula Ia

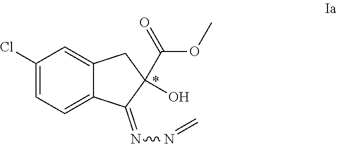 [SMILES: C=NN=C1c2ccc(Cl)cc2CC1(O)C(=O)OC]

with formaldehyde, optionally in the presence of organic solvent selected from the group consisting of toluene, xylene, monochlorobenzene, dichlorobenzene, ethylbenzene, cyclopentylmethyl ether, methyl-tert-butyl ether, benzonitrile and mixtures thereof.

Preferably, the process of preparation of compound VIa performed in the presence of ammonium salt catalyst, which is selected from pyridinium hydrochloride, pyridinium hydrobromide, tetrabutylammonium bromide and the mixtures thereof.

According to an embodiment, the molar ratio of compound formula IIa to formaldehyde is from about 1:1.5 to about 1:10, preferably from about 1:2 to 1:8.

Suitable amount of the catalyst in the process of preparation of compound VIa from compound IIa is from 0.1 to 50 mol %, preferably from 1.0 to 10.0 mol %.

Finally, the reaction mixture containing the resulting compound of formula VIa is worked up. This stage may include water addition, phase separation, filtration, purification, extraction, concentration and recrystallization.

According to the present invention, the compound of formula IIa contacted with formaldehyde at preferred temperature interval from −10 to 180° C., more preferably, from 10 to 55° C.

Another aspect of the present invention provides a process for preparing of compound of formula IV which is racemic or enantiomerically enriched at chiral center*

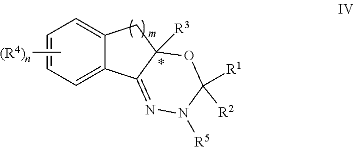

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy

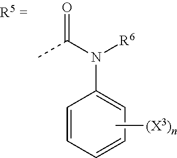

R⁶=hydrogen, CO₂Me, CO₂(R′)
X³=Halogen, OCF₃, SCF₃
n=1-3;
the process comprising the reaction of compound of formula I

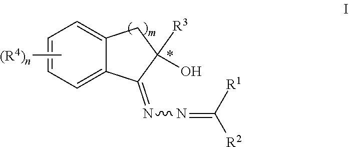

with a compound of formula VII:

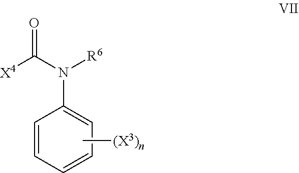

Wherein R⁶=hydrogen, CO₂Me, CO₂(R¹)
n=1-3
X³=Halogen, OCF₃, SCF₃
X⁴=Halogen, tosylate, triflate
in the presence of organic solvent.

In an embodiment the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

According to an embodiment, the molar ratio of compound formula I to compound of formula VII is from 5:1 to about 1:10, preferably from 1.2:1 to 1:3.

Finally, the reaction mixture containing the resulting compound of formula IV is worked up. This stage may include water addition, phase separation, filtration, purification, extraction, adjustment of the pH, concentration and recrystallization. Optimization of these steps lead to purities of resulting compound of formula IV over 95%.

According to an embodiment, the molar ratio of compound formula I to compound of formula VII is from 1.5:1 to about 1:2, preferably from 1.2:1 to 1:1.5 Finally, the reaction mixture containing the resulting compound of formula IV is worked up. This stage may include water addition, phase separation, filtration, purification, extraction, adjustment of the pH, concentration and recrystallization. Optimization of these steps lead to purities of resulting compound of formula IV over 95%.

According to an embodiment, the compound of formula I preferably contacted with the compound of formula VII at temperature interval from −20 to 180° C., more preferably from 45 to 85° C.

According to another embodiment, the compound of formula I contacted with the compound of formula VII at temperature interval from 10 to 100° C., preferably from 55 to 70° C.

In more specific embodiment the present invention provides a process for preparation of compound of formula IVa, which is racemic or enantiomerically enriched at chiral center*

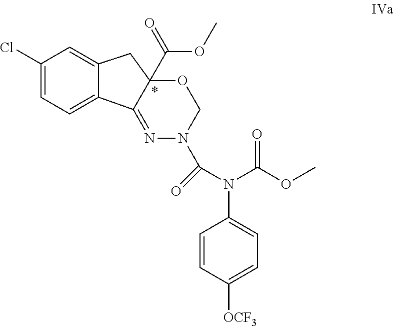 [SMILES: COC(=O)N(C(=O)N1COC2(C(=O)OC)Cc3cc(Cl)ccc3C2=N1)c1ccc(OC(F)(F)F)cc1]

comprising the reaction of compound of formula Ia

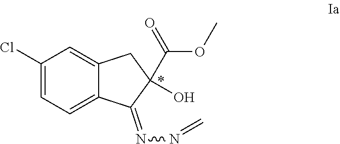 [SMILES: C=NN=C1c2ccc(Cl)cc2CC1(O)C(=O)OC]

with a compound of formula VIIa

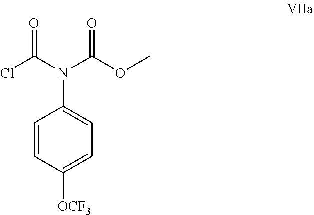 [SMILES: COC(=O)N(C(=O)Cl)c1ccc(OC(F)(F)F)cc1]

in the presence of organic solvent, wherein the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

In a further embodiment the organic aprotic solvent is monochlorobenzene.

According to an embodiment, the molar ratio of compound formula Ia to compound of formula VIIa is from 5:1 to 1:10, preferably from 1.2:1 to 1:3.

Finally, the reaction mixture containing the resulting compound of formula IVa is worked up. This stage may include water addition, phase separation, filtration, purification, extraction, adjustment of the pH, concentration and recrystallization. Optimization of these steps lead to purities of resulting compound of formula IV over 95%.

According to an embodiment, the molar ratio of compound formula Ia to compound of formula VIIa is from 1.5:1 to 1:2, preferably from 1.2:1 to 1:1.5.

Finally, the reaction mixture containing the resulting compound of formula IVa is worked up. This stage may include water addition, phase separation, filtration, purification, extraction, adjustment of the pH, concentration and recrystallization. Optimization of these steps lead to purities of resulting compound of formula IV over 95%.

According to an embodiment, the compound of formula Ia contacted with the compound of formula VIIa at temperature interval from −20 to 180° C., preferably from 45 to 85° C.

According to another embodiment, the compound of formula Ia contacted with the compound of formula Vila at temperature interval from 10 to 100° C., preferably from 55 to 70° C.

According to the present invention, the compound of formula Ia contacted with the compound of formula VIIa at temperature interval from −40 to 80° C., preferably from 50 to 80° C.

In a further embodiment the present invention provides a process for preparing of compound of formula IV which is racemic or enantiomerically enriched at chiral center*

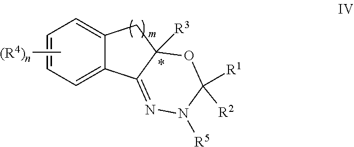

comprising the reaction of compound of formula VI

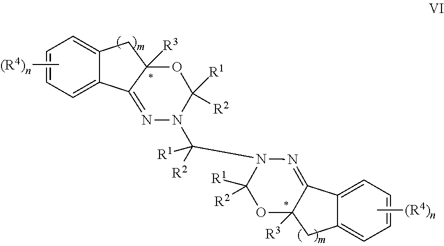

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy
m=1-3
n=1-3
with a compound of formula VII

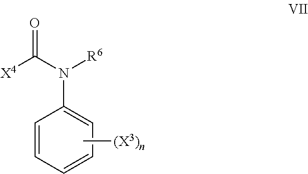

Wherein R⁶=hydrogen, CO₂Me, CO₂(R¹)
n=1-3
X³=Halogen, OCF₃, SCF₃
X⁴=Halogen, tosylate, triflate,
in the presence of organic solvent.

In an embodiment, the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

According to an embodiment, the reaction media include water. In some embodiment due to the presence of water the reaction proceeds in a two-phase system.

According to an embodiment, the molar ratio of compound formula VI to compound of formula VII is from 3:1 to 1:10, preferably from 1:1.5 to 1:5.

Finally, the reaction mixture containing the resulting compound of formula IV is worked up. This stage may include water addition, phase separation, filtration, purification, extraction, adjustment of the pH, concentration and recrystallization. Optimization of these steps lead to purities of resulting compound of formula IV over 95%.

According to the present invention, the compound of formula VI contacted with the compound of formula VII at temperature interval from −20 to 80° C., preferably from 50 to 75° C.

In a further embodiment the present invention provides a process for preparing of compound of formula IVa which is racemic or enantiomerically enriched at chiral center*

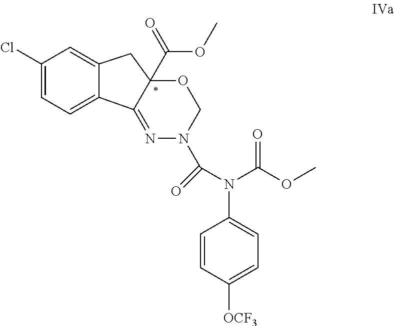

comprising the reaction of compound of formula Via

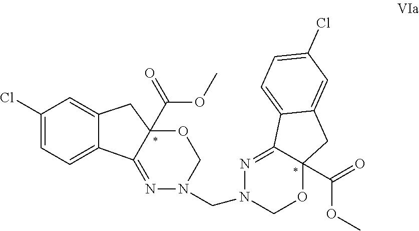

with a compound of formula VIIa

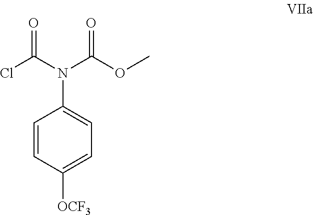 [SMILES: COC(=O)N(C(=O)Cl)c1ccc(OC(F)(F)F)cc1]

in the presence of organic solvent.

In an embodiment, the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

According to an embodiment, the reaction media include water. In some embodiment due to the presence of water the reaction proceeds in a two-phase system.

According to an embodiment, the molar ratio of compound formula VIa to compound of formula VIIa is from 3:1 to 1:10, preferably from 1:1.5 to 1:5.

Finally, the reaction mixture containing the resulting compound of formula IVa is worked up. This stage may include water addition, phase separation, filtration, purification, extraction, adjustment of the pH, concentration and recrystallization. Optimization of these steps lead to purities of resulting compound of formula IV over 95%.

According to the present invention, the compound of formula VIa contacted with the compound of formula VIIa at temperature interval from −20 to 80° C., preferably from 50 to 75° C.

In an additional embodiment the present invention provides a process for preparing of compound of formula IV which is racemic or enantiomerically enriched at chiral center*

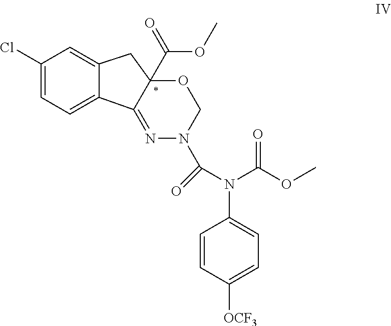

comprising the reaction of compound of formula V

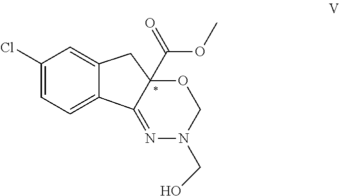

with a compound of formula VII

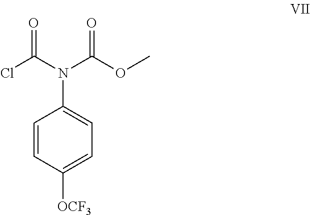 [SMILES: COC(=O)N(C(=O)Cl)c1ccc(OC(F)(F)F)cc1]

in the presence of organic solvent.

In an embodiment, the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

According to an embodiment, the reaction media include water. In some embodiment due to the presence of water the reaction proceeds in a two-phase system.

According to an embodiment, the molar ratio of compound formula V to compound of formula VII is from 3:1 to 1:10, preferably from 1.2:1 to 1:5.

Finally, the reaction mixture containing the resulting compound of formula IV is worked up. This stage may include water addition, phase separation, filtration, purification, extraction, adjustment of the pH, concentration and recrystallization. Optimization of these steps lead to purities of resulting compound of formula IV over 95%.

According to the present invention, the compound of formula V contacted with the compound of formula VII at temperature interval from −20 to 180° C., preferably from 50 to 75° C.

In an additional embodiment the present invention provides a process for preparing of compound of formula IVa which is racemic or enantiomerically enriched at chiral center*

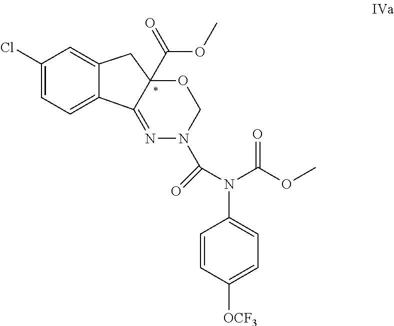

comprising the reaction of compound of formula Va

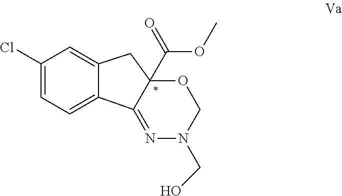

with a compound of formula VIIa

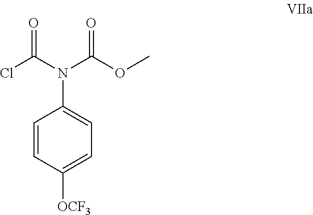 [SMILES: COC(=O)N(C(=O)Cl)c1ccc(OC(F)(F)F)cc1]

in the presence of organic solvent.

In an embodiment, the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

According to an embodiment, the reaction media include water. In some embodiment due to the presence of water the reaction proceeds in a two-phase system.

According to an embodiment, the molar ratio of compound formula Va to compound of formula VIIa is from 3:1 to 1:10, preferably from 1.2:1 to 1:5.

Finally, the reaction mixture containing the resulting compound of formula Va is worked up. This stage may include water addition, phase separation, filtration, purification, extraction, adjustment of the pH to 6-8, concentration and recrystallization. Optimization of these steps lead to purities of resulting compound of formula IV over 95%.

According to the present invention, the compound of formula Va contacted with the compound of formula VIIa at temperature interval from −20 to 80° C., preferably from 50 to 75° C.

In an embodiment, part of the solvent/reaction mixture is evaporated prior to the addition of any compound of formula VII or VIIa.

According to a particular embodiment of the invention the compound of formula IV could be purified by recrystallization from C₁-C₄ alcohols, optionally in the presence of water. In a specific embodiment the compound of formula Va can be purified by recrystallization from isopropanol.

In an embodiment the compound of formula IV which is racemic or enantiomerically enriched at chiral center*

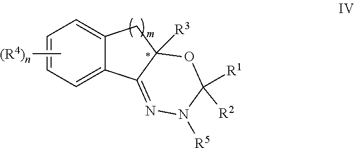

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy

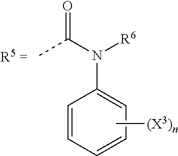

R⁶=hydrogen, CO₂Me, CO₂(R¹)
X³=Halogen, OCF₃, SCF₃
n=1-3;
is prepared from the mixture of the compounds of formula I, V and/or VI comprising the reaction of at list one of the compounds of formula I, V and/or VI:

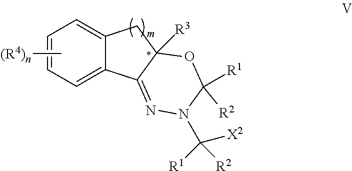

Wherein R¹, R² is independently hydrogen, C₁-C₄-alkyl
R³ is C₁-C₄-alkoxycarbonyl, COOH
R⁴ is halogen, C₁-C₄ alkyl, C₁-C₄alkoxy
X²=OR¹, N(R¹)₂
m=1-3
n=1-3

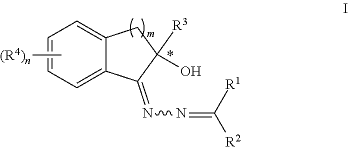

Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3

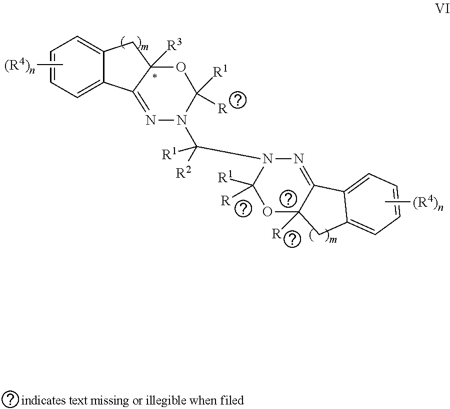

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy
m=1-3
n=1-3
with a compound of formula VII in the presence of organic solvent.

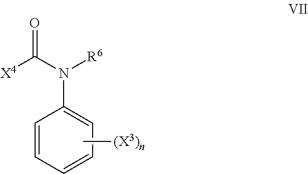

Wherein R⁶=hydrogen, CO₂Me, CO₂(R¹)
n=1-3
X³=Halogen, OCF₃, SCF₃
X⁴=Halogen, triflate, tosylate
in the presence of organic solvent.

In an additional embodiment, a compound of formula IVa which is racemic or enantiomerically enriched at chiral center*

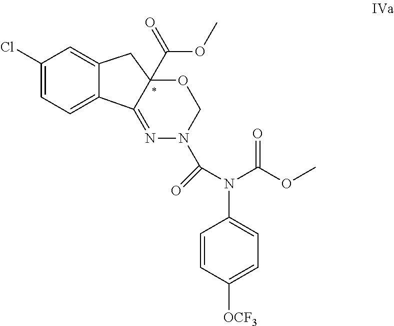 [SMILES: COC(=O)N(C(=O)N1COC2(C(=O)OC)Cc3cc(Cl)ccc3C2=N1)c1ccc(OC(F)(F)F)cc1]

Is prepared by the reaction of at list one of the compounds of formula Ia, Va and/or VIa

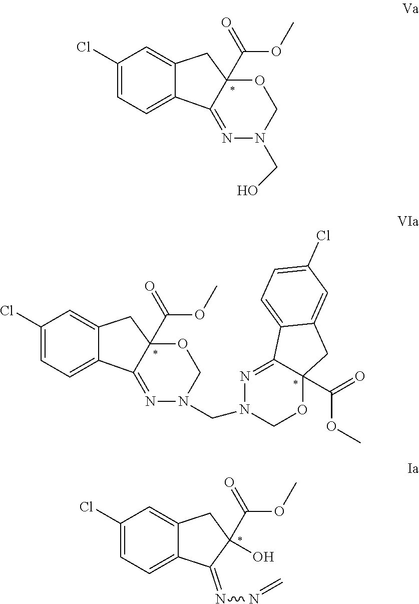

with a compound of formula VIIa

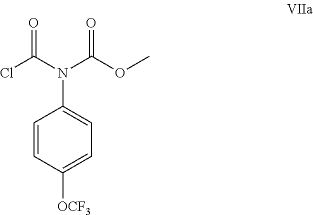 [SMILES: COC(=O)N(C(=O)Cl)c1ccc(OC(F)(F)F)cc1]

in the presence of organic solvent.

According to further embodiment, the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

According to a particular embodiment, the process of preparation of compound of formula IV and/or Va is performed in the presence of water as a co-solvent.

According to an embodiment, the molar ratio of each one of compounds formula VI, I, and/or V in the reactive mixture to compound of formula VII is from 3:1 to 1:10.

According to the present invention, the mixture of compounds of formula VI, I, and/or V contacted with the compound of formula VII at temperature interval from −20 to 80° C., preferably from 50 to 75° C.

Finally, the reaction mixture containing the resulting compound of formula IV is worked up. This stage may include water addition, phase separation, filtration, purification, extraction, adjustment of the pH, concentration and recrystallization. Optimization of these steps lead to purities of resulting compound of formula IV over 95%.

According to an embodiment the compound of formula IV which is racemic or enantiomerically enriched at chiral center*

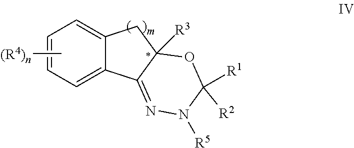

Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy

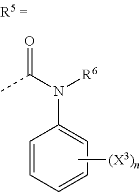

R⁶=hydrogen, CO₂Me, CO₂(R′)
X³=Halogen, OCF₃, SCF₃
n=1-3;
is prepared by a telescopic process comprising the steps of: a) preparation of compound I, or V, or VI or the mixture thereof b) reaction of compound I, or V, or VI or the mixture thereof with compound of formula VII in the presence of organic solvent. The organic solvent suitable for telescopic process of preparation of compound of formula IV aprotic solvent such as toluene, xylene, monochlorobenzene, dichlorobenzene, ethylbenzene, cyclopentylmethyl ether, methyl-tert-butyl ether, benzonitrile and mixtures thereof.

Telescoped solutions of intermediates I, V, VI or the mixtures thereof can be extracted, filtered (as long as the desired intermediate I, V, VI or the mixtures thereof remains in the filtrate), and solvent exchanged, but the corresponding intermediate is ultimately held in solution and carried forward to the subsequent transformation.

Another aspect of the present invention provides a novel compound of formula VIII (methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate) which is racemic or enantiomerically enriched at chiral center*

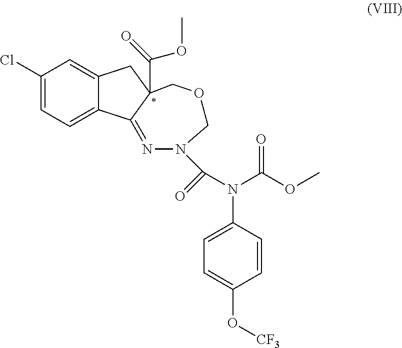 [SMILES: COC(=O)N(C(=O)N1COCC2(C(=O)OC)Cc3cc(Cl)ccc3C2=N1)c1ccc(OC(F)(F)F)cc1]

In an embodiment the compound of formula VIII is prepared by a process according to the following scheme 1:

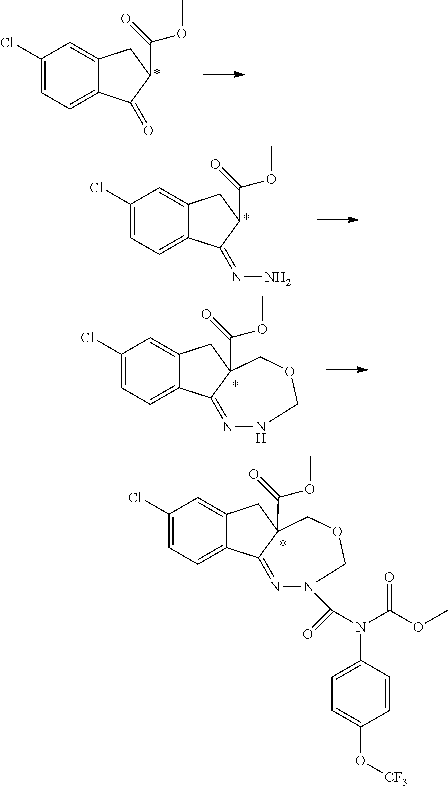

### Scheme 1: Preparation of methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate (Compound of Formula VIII)

The aforementioned process comprising: a) reacting the mixture of methyl (S)-5-chloro-2-hydroxy-1-oxo-2,3-dihydro-1H-indene-2-carboxylate and (1H-Indene-2-carboxylic acid, 5-chloro-2,3-dihydro-1-oxo-, methyl ester) with hydrazine hydrate in the presence of methanol and acetic acid to prepare the mixture of methyl (S)-5-chloro-1-hydrazineylidene-2-hydroxy-2,3-dihydro-1H-indene-2-carboxylate and methyl 5-chloro-1-hydrazineylidene-2,3-dihydro-1H-indene-2-carboxylate, b) reacting the mixture of methyl (S)-5-chloro-1-hydrazineylidene-2-hydroxy-2,3-dihydro-1H-indene-2-carboxylate and methyl 5-chloro-1-hydrazineylidene-2,3-dihydro-1H-indene-2-carboxylate prepared in a) with formaldehyde in the presence of pyridine hydrochloride to prepare the mixture of methyl (S)-5-chloro-2-hydroxy-1-(methylenehydrazineylidene)-2,3-dihydro-1H-indene-2-carboxylate and methyl 8-chloro-2,6-dihydro-3H-indeno[1,2-e][1,3,4]oxadiazepine-5a(5H)-carboxylate, c) reacting the mixture of methyl (S)-5-chloro-2-hydroxy-1-(methylenehydrazineylidene)-2,3-dihydro-1H-indene-2-carboxylate and methyl 8-chloro-2,6-dihydro-3H-indeno[1,2-e][1,3,4]oxadiazepine-5a(5H)-carboxylate, prepared in step b) with methyl N-carbonochloridoyl-N-[4-(trifluoromethoxy)phenyl]carbamate to produce final compound of formula VIII (methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate).

Finally, the reaction mixture containing the resulting compound of formula VIII is worked up. This stage may include water addition, phase separation, filtration, purification, extraction, adjustment of the pH to 6-8, concentration and recrystallization. Optimization of these steps lead to purities of resulting compound of formula VIII over 78% purity.

According to another embodiment the compound of formula VIII can be purified by recrystallization from the mixture of isopropanol/water, wherein the ratio of isopropanol to water is 3:1.

According to further embodiment, the compounds I, Ia, V, Va, VI, VIa, and VIII exist as corresponding acid salts or complex metal salts. Suitable acid salts are those formed with inorganic, organic acids or metal salts. Suitable inorganic acids are, for example, hydrochloric acid, sulfuric acid, phosphoric acid, nitric acid etc. Suitable organic acids are, for example, acetic acid, propionic acid, citric acid, tartaric acid etc. Suitable complex metal salts including Li, Na, Mg, Zn, Ni, Cu and Fe salts.

The following examples illustrate the practice of the present invention in some of its embodiments but should not be construed as limiting the scope of the invention. Other embodiments will be apparent to one skilled in the art from consideration of the specification and examples. It is intended that the specification, including the examples, is considered exemplary only without limiting the scope and spirit of the invention.

### EXAMPLES

### Example 1: Preparation of methyl (S)-5-chloro-1-hydrazineylidene-2-hydroxy-2,3-dihydro-1H-indene-2-carboxylate (Compound II)

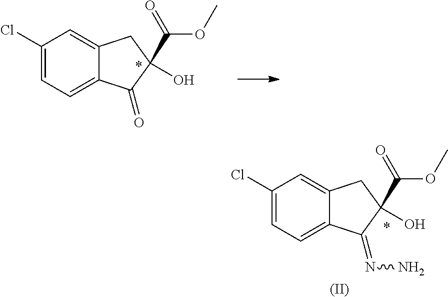 [SMILES: COC(=O)[C@]1(O)Cc2cc(Cl)ccc2C1=O ; COC(=O)[C@]1(O)Cc2cc(Cl)ccc2C1=NN ; "(II)"]

To a stirred solution of acetic acid (AcOH) (112.5 gr) in 2.4 L MCB (monochlorobenzene) at 10° C. was added dropwise hydrazine hydrate (N₂H₄·H₂O) 64% (84.3 gr). The mixture was stirred for 15 min and then 0.6 L of methanol were added. Then the reaction mixture was stirred for 5 min and solid methyl (S)-5-chloro-2-hydroxy-1-oxo-2,3-dihydro-1H-indene-2-carboxylate in S/R isomer ratio of 9:1 (300 gr) was added in one portion. The reaction mixture was heated to 60° C. and stirred for 4 hours. After that the reaction mixture was cooled to 45-50° C. and methanol was removed under reduced pressure. MCB (monochlorobenzene) was added to reach the mass prior to evaporation and the organic layer was washed with 0.75 L of water. This organic phase containing methyl (S)-5-chloro-1-hydrazineylidene-2-hydroxy-2,3-dihydro-1H-indene-2-carboxylate (II) was taken to the next step without additional treatment (90% yield).

### Example 2: Preparation of methyl (S)-5-chloro-2-hydroxy-1-(methylenehydrazineylidene)-2,3-dihydro-1H-indene-2-carboxylate (Compound I)

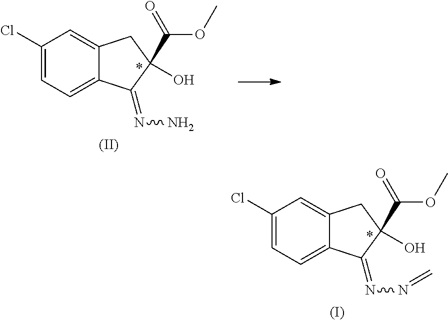

To the stirred solution of methyl (S)-5-chloro-1-hydrazineylidene-2-hydroxy-2,3-dihydro-1H-indene-2-carboxylate (compound IIa) at 15-18° C., formaldehyde 37% (containing 3% MeOH) (273.6 gr) was added in three portions followed by addition of Pyridine hydrochloride (6.5 gr). The reaction mixture was heated to 25° C. and continued to stir for 2 h. Then the reaction mixture was washed with 1.5 L H₂O, and 10% of the solvent was distilled out from the organic phase under reduces pressure. Resulting MCB (monochlorobenzene) solution of methyl (S)-5-chloro-2-hydroxy-1-(methylenehydrazineylidene)-2,3-dihydro-1H-indene-2-carboxylate (compound I) was taken to the next step without additional treatment.

### Example 3: Preparation of Indoxacarb (Compound IV)

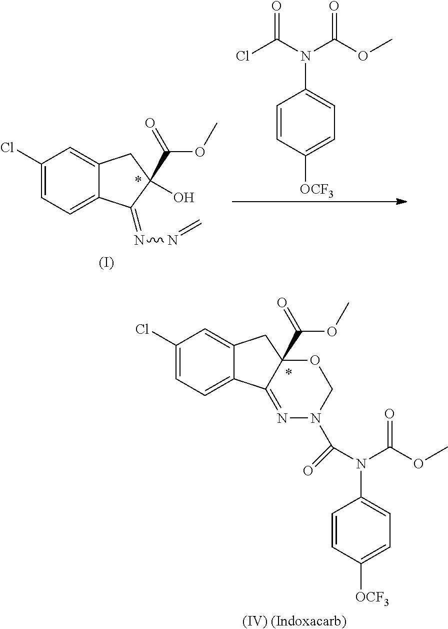

To the fresh MCB (monochlorobenzene) solution of methyl (S)-5-chloro-2-hydroxy-1-(methylenehydrazineylidene)-2,3-dihydro-1H-indene-2-carboxylate (compound I), prepared in the previous step, Methyl N-carbonochloridoyl-N-[4-(trifluoromethoxy)phenyl]carbamate (compound VII) (334 gr) was added and the resulting reaction mixture was heated to 60° C. for 1.5 hours, then a solution of 5% aqueous NaHCO₃ was added to maintain pH 3 and stirred for additional 2 hours. After that, the reaction mixture was cooled to room temperature, the pH was adjusted to 7 (using a solution of 5% aqueous NaHCO₃) and the organic phase was separated. The solvent was removed under vacuum to give crude indoxacarb (compound IV) in 63% yield with S/R ratio of 9:1.

### Example 4: Preparation of methyl (S)-7-chloro-2-(hydroxymethyl)-2,5-dihydroindeno[1,2-e][1,3,4]oxadiazine-4a(3H)-carboxylate (Compound V)

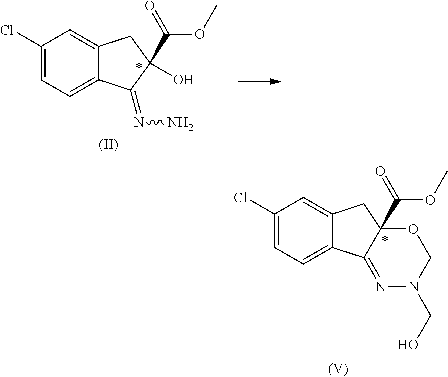 [SMILES: COC(=O)[C@]1(O)Cc2cc(Cl)ccc2C1=NN ; "(II)" ; COC(=O)[C@]12Cc3cc(Cl)ccc3C1=NN(CO)CO2 ; "(V)"]

The solution of methyl (S)-5-chloro-1-hydrazineylidene-2-hydroxy-2,3-dihydro-1H-indene-2-carboxylate (II) prepared in Example 1 was stirred at 15° C., and formaldehyde 37% (3% MeOH) (13.56 Kg) was added in three portions followed by addition of pyridine hydrochloride (0.05 eq., 65 gr). The reaction mixture is heated to 25° C. and continued to stir overnight to form a solution of methyl (S)-7-chloro-2-(hydroxymethyl)-2,5-dihydroindeno[1,2-e][1,3,4]oxadiazine-4a(3H)-carboxylate (compound V) in 30% yield.

### Example 5: Preparation of Indoxacarb (Compound IV)

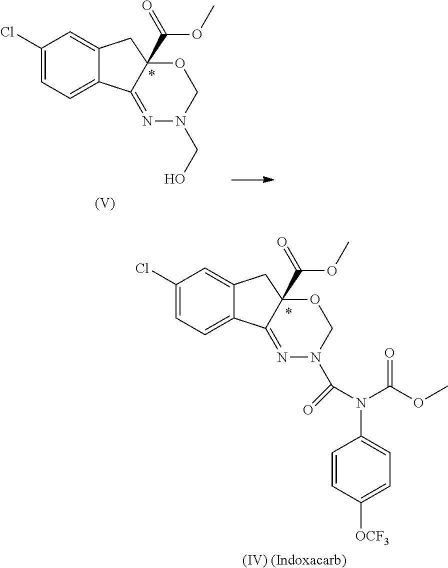 [SMILES: COC(=O)[C@]12Cc3cc(Cl)ccc3C1=NN(CO)CO2 ; "(V)" ; COC(=O)N(C(=O)N1CO[C@@]2(C(=O)OC)Cc3cc(Cl)ccc3C2=N1)c1ccc(OC(F)(F)F)cc1 ; "(IV) (Indoxacarb)"]

1 L of the monochlorobenzene solution containing 34 g of methyl (S)-7-chloro-2-(hydroxymethyl)-2,5-dihydroindeno[1,2-e][1,3,4]oxadiazine-4a(3H)-carboxylate (compound Va) prepared in Example 4, was washed with 0.5 L H₂O, and about 10% of the solvent is distilled out from the organic phase under reduced pressure. After that the resulting mixture was heated to 50° C. and methyl N-carbonochloridoyl-N-[4-(trifluoromethoxy)phenyl]carbamate (compound VII) (40 gr) was added. Then the reaction mixture was stirred for 10 min. then 0.3 L of water was added and the reaction mixture was stirred for 2 h and then cooled to room temperature and the pH was adjusted to 7 (using a solution of 5% aqueous NaHCO₃) and the organic phase was separated. The solvent was removed under vacuum to give crude Indoxacarb (compound IV) in 65% yield with S/R ratio of 9:1.

### Example 6: Preparation of dimethyl 2,2′-methylene(4aS,4a'S)-bis(7-chloro-2,5-dihydroindeno[1,2-e][1,3,4]oxadiazine-4a(3H)-carboxylate) (Compound VI)

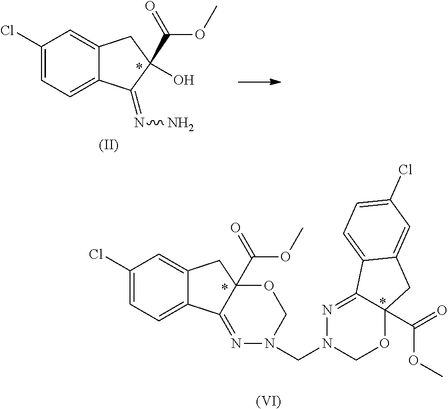 [SMILES: COC(=O)[C@]1(O)Cc2cc(Cl)ccc2C1=NN ; "(II)" ; COC(=O)C12Cc3cc(Cl)ccc3C1=NN(CN1COC3(C(=O)OC)Cc4cc(Cl)ccc4C3=N1)CO2 ; "(VI)"]

To the mixture containing 6.9 g of Dimethyl(methylene)iminium chloride (freshly prepared according to common methods) in 20 ml monochlorobenzene (, methyl (S)-5-chloro-1-hydrazineylidene-2-hydroxy-2,3-dihydro-1H-indene-2-carboxylate (II) (9.525 g in 80 ml monochlorobenzene) was added and the resulting reaction mixture was heated to 55° C. and stirred for ˜16.5 h. Then 90 ml of NaHCO₃(a_{q}) (saturated) was added and the mixture was washed with two portions of 120 ml of water. The aqueous phase was extracted with 10 ml of monochlorobenzene (MCB) and the organic phase was concentrated under reduced pressure to afford dimethyl 2,2′-methylene(4aS,4a'S)-bis(7-chloro-2,5-dihydroindeno[1,2-e][1,3,4]oxadiazine-4a(3H)-carboxylate) (compound VI), which then purified by preparative chromatography (water:acetonitrile gradient, YMC-Actus Trart C18 column) to provide 1.82 g (96.7% purity) of dimethyl 2,2′-methylene(4aS,4a'S)-bis(7-chloro-2,5-dihydroindeno[1,2-e][1,3,4]oxadiazine-4a(3H)-carboxylate) (compound VI).

### Example 7: Preparation of Indoxacarb (Compound IV)

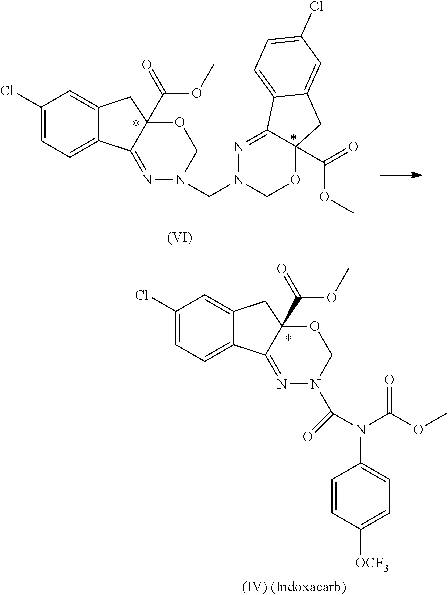 [SMILES: COC(=O)C12Cc3cc(Cl)ccc3C1=NN(CN1COC3(C(=O)OC)Cc4cc(Cl)ccc4C3=N1)CO2 ; "(VI)" ; COC(=O)N(C(=O)N1CO[C@@]2(C(=O)OC)Cc3cc(Cl)ccc3C2=N1)c1ccc(OC(F)(F)F)cc1 ; "(IV) (Indoxacarb)"]

The 150 ml of the solution containing 4 g of dimethyl 2,2′-methylene(4aS,4a'S)-bis(7-chloro-2,5-dihydroindeno[1,2-e][1,3,4]oxadiazine-4a(3H)-carboxylate) (compound VI) in MCB (monochlorobenzene) prepared in Example 6 was washed with 50 ml of water and the organic phase was concentrated under reduced pressure at 30° C. over 1 h.

Then the resulting solution was heated to 60° C. and methyl N-carbonochloridoyl-N-[4-(trifluoromethoxy)phenyl]carbamate (VII) (6 gr) was added and the reaction mixture was stirred for 10 min and after addition of 40 ml of water stirred for additional 2 hours, then is cooled to room temperature, the pH was adjusted to 7 (using a solution of 5% aqueous NaHCO₃) and the organic phase was separated. After that the solvent was removed under vacuum to give crude Indoxacarb (compound IV) in 67% yield with S/R ratio of 9:1.

### Example 8: Preparation of methyl (S)-5-chloro-1-hydrazineylidene-2-hydroxy-2,3-dihydro-1H-indene-2-carboxylate (Compound II)

### Step 1

To a stirred solution of acetic acid (112.5 gr) in 2.4 L MCB (monochlorobenzene) at 10-20° C., N₂H₄·H₂O 64% (84.3 gr) was added dropwise and then 0.6 L MeOH were added and stirred. methyl (S)-5-chloro-2-hydroxy-1-oxo-2,3-dihydro-1H-indene-2-carboxylate in S/R ratio of 9:1 (300 gr) was added to the resulting mixture and the reaction admixture was heated to 66° C. and stirred for 5 h. Then the reaction mixture was cooled and concentrated under reduced pressure to remove methanol. After methanol removal, the organic phase was washed with about 0.75 L of water to afford methyl (S)-5-chloro-1-hydrazineylidene-2-hydroxy-2,3-dihydro-1H-indene-2-carboxylate (II) which was taken to the next step without additional treatment (90% yield).

### Step 2: Preparation of methyl (S)-5-chloro-2-hydroxy-1-(methylenehydrazineylidene)-2,3-dihydro-1H-indene-2-carboxylate (Compound I)

The stirred solution of methyl (S)-5-chloro-1-hydrazineylidene-2-hydroxy-2,3-dihydro-1H-indene-2-carboxylate (II) prepared in Step 1 in monochlorobenzene was kept at 18° C., and formaldehyde 37% (containing 3% MeOH) (273.6 gr) was added and after that pyridine hydrochloride (6.5 gr) was inserted to the reaction mixture which then was heated to 25° C. and stirred for additional 5 hours. Then the reaction mixture was washed with water, and the organic phase was concentrated under reduced pressure (about 10% of the solvent was distilled out) to give MCB (monochlorobenzene) solution of methyl (S)-5-chloro-2-hydroxy-1-(methylenehydrazineylidene)-2,3-dihydro-1H-indene-2-carboxylate (I) which was taken to the next step without separation.

### Step 3: Preparation of Indoxacarb (Compound IV)

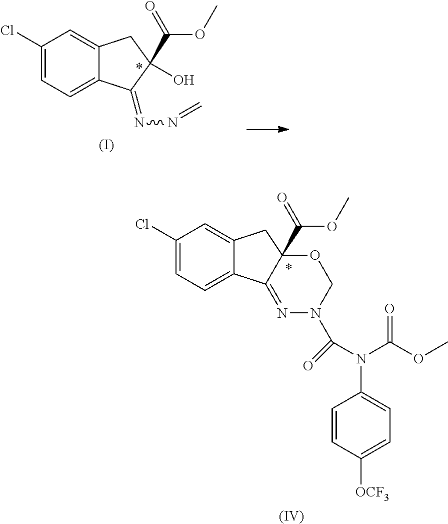

To the MCB (monochlorobenzene) solution of methyl (S)-5-chloro-2-hydroxy-1-(methylenehydrazineylidene)-2,3-dihydro-1H-indene-2-carboxylate (I), prepared in the previous step, Methyl N-carbonochloridoyl-N-[4-(trifluoromethoxy)phenyl]carbamate (compound VII) (334 gr) was added and the reaction was heated to 60° C. for 2 hours. Then 0.5 L of water was added and the reaction was stirred for additional 2 hours. After that the organic phase was separated, washed with water and the pH was adjusted to 7 (using aqueous solution of 5% NaHCO₃) and the organic phase was separated. The solvent was removed under vacuum to give crude indoxacarb (compound IV) in 65% yield.

### Example 9: Purification of Compound IV (Indoxacarb) by Recrystallization

Crude compound IV (Indoxacarb) (650-700 gr) obtained by any of the preceding Examples was dissolved at 82° C. in 1.7 L isopropanol and then cooled to room temperature during 1.5 hours. After that, 38 ml of water and 8 gr Indoxacarb (97.5% purity) were added to said solution and the mixture was cooled to 0° C. for the period of 4 hours. Then the mixture was stirred at 0° C. overnight and the obtained solid material was filtered out, washed with isopropanol and dried to afford 320-350 gr of compound IV (Indoxacarb) 97.5% purity with S/R ratio of 9:1.

### Example 10: Preparation of methyl (S)-5-chloro-1-hydrazineylidene-2-hydroxy-2,3-dihydro-1H-indene-2-carboxylate (Compound II)

### Step 1

To a stirred solution of acetic acid (18.8 gr) in 481 gr MCB (monochlorobenzene) and 79.3 gr MeOH (methanol) at 10-20° C., N₂H₄·H₂O 64% (14.4 gr) was added dropwise and the mixture was stirred for 30 min. Methyl (S)-5-chloro-2-hydroxy-1-oxo-2,3-dihydro-1H-indene-2-carboxylate in S/R ratio of 9:1 (50 gr) was added to the resulting mixture. The reaction mixture was heated to 60° C. and stirred for 3 hours. Then the reaction mixture was cooled and concentrated under reduced pressure at 40° C. for the removal of MeOH. After MeOH removal, 60 gr MCB was added and the organic layer was washed with 135 mL of water at 25° C. to afford methyl (S)-5-chloro-1-hydrazineylidene-2-hydroxy-2,3-dihydro-1H-indene-2-carboxylate (II). The aqueous phase was extracted with 40 gr of MCB. Methyl (S)-5-chloro-1-hydrazineylidene-2-hydroxy-2,3-dihydro-1H-indene-2-carboxylate (II) was taken to the next step without additional treatment (90% yield).

### Step 2: Preparation of methyl (S)-5-chloro-2-hydroxy-1-(methylenehydrazineylidene)-2,3-dihydro-1H-indene-2-carboxylate (Compound I)

The stirred solution of Methyl (S)-5-chloro-1-hydrazineylidene-2-hydroxy-2,3-dihydro-1H-indene-2-carboxylate (II) prepared in Step 1 in MCB was heated to 60° C. Formaldehyde 37% (containing 3% MeOH) (32.4 gr) was added to the reaction mixture and stirred for 2 hours. Then the reaction mixture was washed with 270 mL water, and the organic phase was concentrated under reduced pressure (about 10% of the solvent was distilled out) to give MCB solution of methyl (S)-5-chloro-2-hydroxy-1-(methylenehydrazineylidene)-2,3-dihydro-1H-indene-2-carboxylate (I) which was taken to the next step without additional treatment.

### Step 3: Preparation of Indoxacarb (Compound IV)

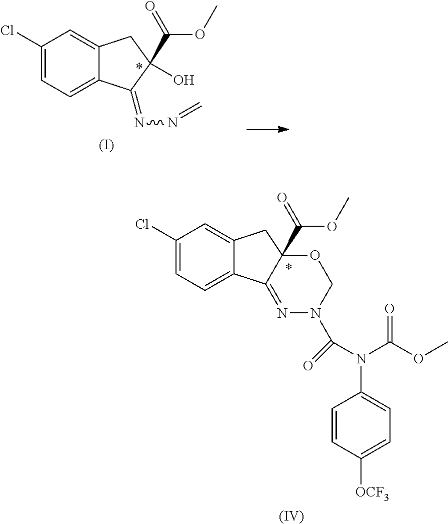

To the MCB solution of Methyl (S)-5-chloro-2-hydroxy-1-(methylenehydrazineylidene)-2,3-dihydro-1H-indene-2-carboxylate (I), prepared in the previous step, Methyl N-carbonochloridoyl-N-[4-(trifluoromethoxy)phenyl]carbamate (compound VII) (54 gr) was added and the reaction was heated to 60° C. for 3 hours. Then 190 mL of water was added dropwise for 1 hour. After phase separation, the aqueous phase was extracted with 40 gr of MCB. The pH of the organic phase was adjusted to 7 using aqueous solution of 5% NaHCO₃ following phase separation. The solvent was removed under vacuum to give crude Indoxacarb (compound IV) in 67% yield.

### PC of Methyl 8-chloro-2,6-dihydro-3H-indeno[1,2-e][1,3,4]oxadiazepine-5a(5H)-carboxylate (VIII)

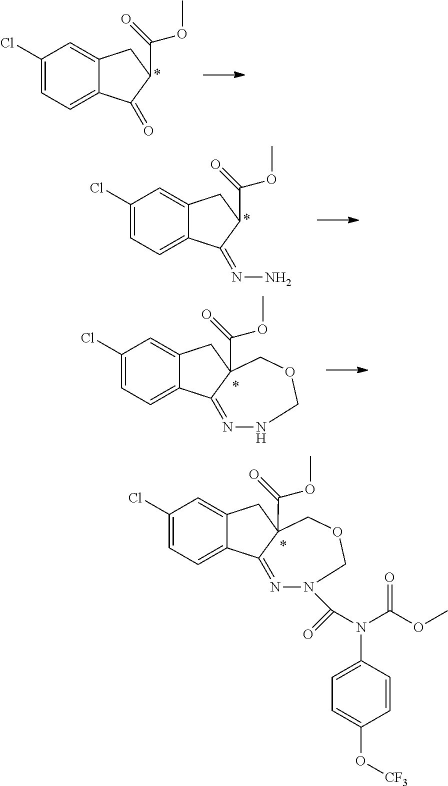

Step 1: Preparation of methyl 5-chloro-1-hydrazineylidene-2,3-dihydro-1H-indene-2-carboxylate:

To a stirred solution of acetic acid (AcOH) (18.8 gr, 1.5 equiv.), 79.3 gr of methanol and 440 gr of monochlorobenzene (MCB) at 10° C. was added dropwise hydrazine hydrate (N₂H₄H₂O) 64% (14.2 gr, 1.35 equiv.). The resulting mixture was stirred for 10 min and solid methyl (S)-5-chloro-2-hydroxy-1-oxo-2,3-dihydro-1H-indene-2-carboxylate (50 gr) and solid (1H-Indene-2-carboxylic acid, 5-chloro-2,3-dihydro-1-oxo-, methyl ester) (5 gr) were added. The reaction mixture was heated to 60° C. and stirred for 3 hours.

After that the reaction mixture was cooled to 40° C. and methanol was removed under reduced pressure, then the remaining solution was washed with 125 gr of water. The organic phase containing methyl (S)-5-chloro-1-hydrazineylidene-2-hydroxy-2,3-dihydro-1H-indene-2-carboxylate and [methyl 5-chloro-1-hydrazineylidene-2,3-dihydro-1H-indene-2-carboxylate]was taken to the next step without additional treatment.

### Step 2: Preparation of methyl 8-chloro-2,6-dihydro-3H-indeno[1,2-e][1,3,4]oxadiazepine-5a(5H)-carboxylate

To the stirred solution prepared in previous Step 1 containing methyl (S)-5-chloro-1-hydrazineylidene-2-hydroxy-2,3-dihydro-1H-indene-2-carboxylate/methyl 5-chloro-1-hydrazineylidene-2,3-dihydro-1H-indene-2-carboxylate at 10° C., formaldehyde 37% (45.6 gr, 2.8 equiv.) was added in three portions followed by addition of pyridine hydrochloride (0.05 equiv, 1.1 gr). The resulting mixture was heated to 25° C. stirred for 2 hours then washed with 250 ml of water. Organic phase was separated and traces of methanol and water were removed from the organic phase under reduces pressure at 60° C., at 50 mbar (during 1 hour). The resulting solution of methyl (S)-5-chloro-2-hydroxy-1-(methylenehydrazineylidene)-2,3-dihydro-1H-indene-2-carboxylate/[methyl 8-chloro-2,6-dihydro-3H-indeno[1,2-e][1,3,4]oxadiazepine-5a(5H)-carboxylate]was taken to the next step without additional treatment.

### Step 3: Preparation of methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate

To the monochlorobenzene solution of methyl (S)-5-chloro-2-hydroxy-1-(methylenehydrazineylidene)-2,3-dihydro-1H-indene-2-carboxylate and methyl 8-chloro-2,6-dihydro-3H-indeno[1,2-e][1,3,4]oxadiazepine-5a(5H)-carboxylate, prepared in the previous step 2, was added methyl N-carbonochloridoyl-N-[4-(trifluoromethoxy)phenyl]carbamate (58.2 gr, 1 equiv.) and the reaction was heated to 60° C. for 1.5 hours. Then 400 gr of water was added to the reaction mixture and the resulting mixture was stirred for 2 hours, fallow by phase separation then washed with 31 gr of 5% aqueous NaHCO₃ and with 308 gr of water. The organic phase was separated and the solvent was evaporated to afford crude mixture of Indoxacarb and methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate

### Purification:

The entire crude product of previous step 3 was dissolved in the mixture of 135 mL of isopropanol (IPA) and 8 gr water at 50° C., and then cooled to 25° C. during 1.5 h, followed by seeding of 0.8 gr Indoxacarb (>94% purity). Then the resulting mixture was stirred for 3 hours at 25° C., then cooled to −3° C. during 10 hours, filtered with centrifuge, washed with cold isopropanol, and dried over vacuum at 45° C. during 4 hours to afford a mixture of Indoxacarb and 7.53 g of methyl 8-chloro-2,6-dihydro-3H-indeno[1,2-e][1,3,4]oxadiazepine-5a(5H)-carboxylate solid that contained 4.2% of methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate.

This solid was separated using preparative chromatography (water:acetonitrile gradient, YMC-Actus Trart C18 column) to afford 0.17 g of methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate (79.7% purity) as off white solid.

1. A di-imine of formula I which is racemic or enantiomerically enriched at chiral center*: 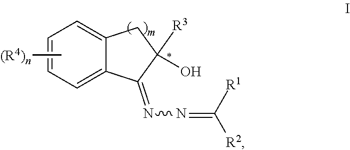
Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3

2. A di-imine of formula I, which is racemic or enantiomerically enriched at chiral center*, wherein R¹ is H, R² is H, R³ is CO₂Me, R⁴ is Cl, n is 1, m is 1:

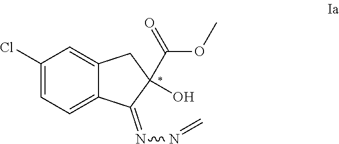

3. A process for preparation of a di-imine of formula I which is racemic or enantiomerically enriched at chiral center*, comprising reaction of compound of formula II 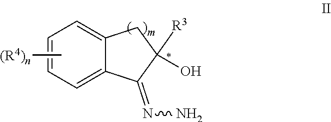
Wherein R³ is C₁-C₄-alkoxycarbonyl, COOH
R⁴ is halogen, C₁-C₄ alkyl, C₁-C₄ alkoxy
m=1-3
n=1-3
with compound of formula III or/and compound of formula IIIa 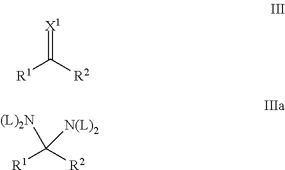
Wherein X¹=O, ⁺NL₂X⁻, L is hydrogen, C₁-C₄ alkyl, X is halogen, R¹, R² are independently hydrogen, C₁-C₄-alkyl, in the presence of organic solvent and optionally, in the presence of catalyst.

4. The process according to claim 3 wherein the catalyst is ammonium salt.

5. The process according to claim 3 wherein the catalyst is ammonium salt selected from the group consisting of pyridinium hydrochloride, pyridinium acetate, pyridinium triflate, pyridinium hydrobromide, tetrabutylammonium bromide, tetrabutylammonium chloride, tetrabutylammonium hydrogen sulfate, tetrabutylammonium iodide, tetraethylammonium bromide, tetraethylammonium chloride, tetraethylammonium hydrogen sulfate, tetraethylammonium iodide, tetramethylammonium bromide, tetramethylammonium chloride, tetramethylammonium hydrogen sulfate, tetramethylammonium iodide, tetrapropylammonium bromide, tetrapropylammonium chloride, tetrapropylammonium hydrogen sulfate, tetrapropylammonium iodide, tetraoctylammonium bromide, tetraoctylammonium chloride, tetraoctylammonium hydrogen sulfate, tetraoctylammonium iodide and the mixtures thereof.

6. The process according to claim 3 wherein the organic solvent is an aprotic solvent.

7. The process according to claim 6 wherein the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and the mixtures thereof.

8. A process for preparation of compound of formula Ia which is racemic or enantiomerically enriched at chiral center* 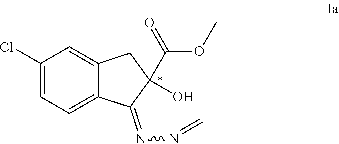
comprising the reaction of compound of formula IIa, 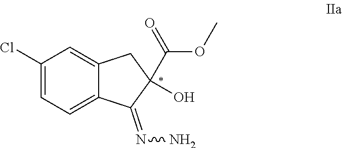
with formaldehyde, and/or with N,N-Dimethylmethyleneiminium chloride (CAS 30354-18-8) and/or with tetramethylmethanediamine in the presence of organic solvent selected from the group consisting of toluene, xylene, monochlorobenzene, dichlorobenzene, ethylbenzene, cyclopentylmethyl ether, methyl-tert-butyl ether, benzonitrile and mixtures thereof, optionally in the presence of ammonium salt.

9. The process according to claim 8 wherein ammonium salt is present.

10. The process according to claim 9 wherein the ammonium salt is selected from pyridinium hydrochloride, pyridinium hydrobromide, tetrabutylammonium bromide and the mixtures thereof.

11. A compound of formula V, which is racemic or enantiomerically enriched at chiral center*: 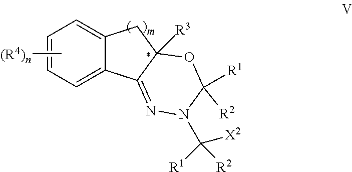
Wherein R¹, R² is independently hydrogen, C₁-C₄-alkyl
R³ is C₁-C₄-alkoxycarbonyl, COOH
R⁴ is halogen, C₁-C₄ alkyl, C₁-C₄alkoxy
X²=OR¹, N(R¹)₂
m=1-3
n=1-3

12. A compound of formula V, which is racemic or enantiomerically enriched at chiral center* wherein R¹ is H, R² is H, R³ is CO₂Me, R⁴ is Cl, n is 1, m is 1, X² is OH:

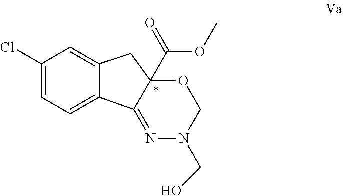

13. A process for preparation of compound of formula V, which is racemic or enantiomerically enriched at chiral center*, 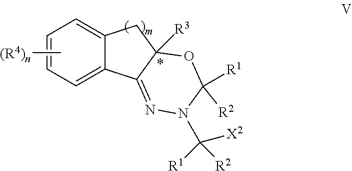
Wherein R¹, R² is independently hydrogen, C₁-C₄-alkyl
R³ is C₁-C₄-alkoxycarbonyl, COOH
R⁴ is halogen, C₁-C₄ alkyl, C₁-C₄ alkoxy
X²=OR¹, N(R¹)₂
m=1-3
n=1-3
comprising reaction of compound of formula I 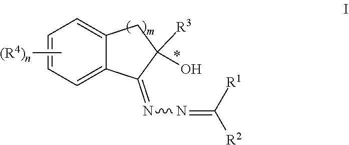
Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3
with compound of formula III or/and compound of formula IIIa 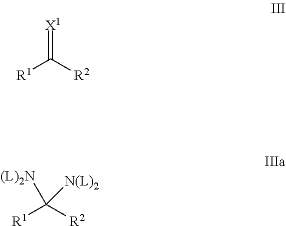
Wherein X¹=O, ⁺NL₂X⁻, L is hydrogen, C₁-C₄ alkyl, X is halogen, R¹, R² are independently hydrogen, C₁-C₄ alkyl, in the presence of organic solvent and, optionally, in the presence of catalyst.

14. The process according to claim 13 wherein the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

15. A process of preparing of compound of formula Va, which is racemic or enantiomerically enriched at chiral center*, 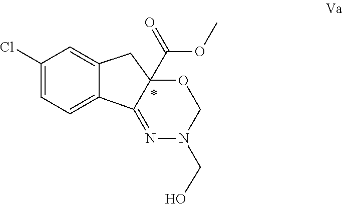 [SMILES: COC(=O)C12Cc3cc(Cl)ccc3C1=NN(CO)CO2]
the process comprising the reaction of compound of formula Ia 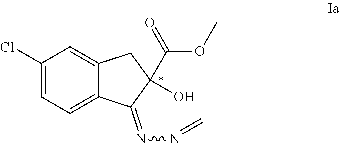
with formaldehyde in the presence of organic solvent, and optionally in the presence of catalyst.

16. The process according to claim 15 wherein the organic solvent is an aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

17. The process according to claim 16 wherein the catalyst is present.

18. The process according to claim 17 wherein the catalyst is an ammonium salt selected from pyridinium hydrochloride, pyridinium hydrobromide, tetrabutylammonium bromide and the mixtures thereof.

19. A compound of formula VI, which is racemic or enantiomerically enriched at chiral center*: 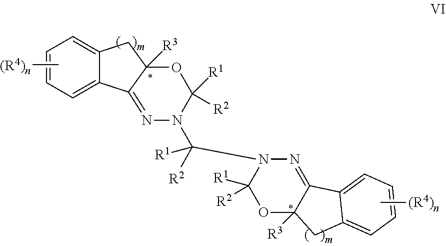
Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy
m=1-3
n=1-3

20. A compound of formula VIa, which is racemic or enantiomerically enriched at chiral center wherein R¹ is H, R² is H, R³ is CO₂Me, R⁴ is Cl, n is 1, m is 1:

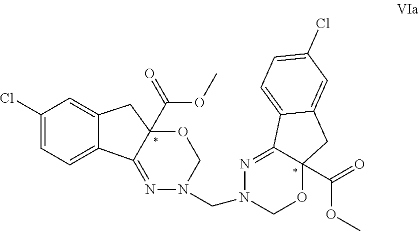

21. A process of preparing of compound of formula VI, which is racemic or enantiomerically enriched at chiral center* 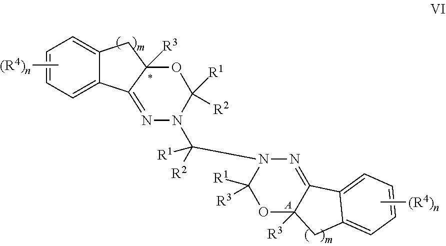
the process comprising the reaction of compound of formula I 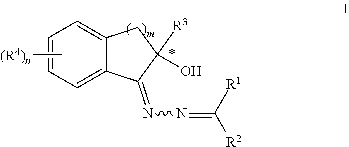
with a compound of formula V 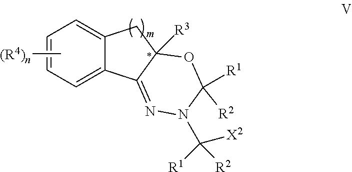
Wherein R¹, R² is independently hydrogen, C₁-C₄-alkyl
R³ is C₁-C₄-alkoxycarbonyl, COOH
R⁴ is halogen, C₁-C₄ alkyl, C₁-C₄ alkoxy
X²=OR¹, N(R¹)₂
m=1-3
n=1-3
in the presence of organic solvent and optionally, in the presence of catalyst.

22. A process of preparing of compound of formula VI which is racemic or enantiomerically enriched at chiral center* 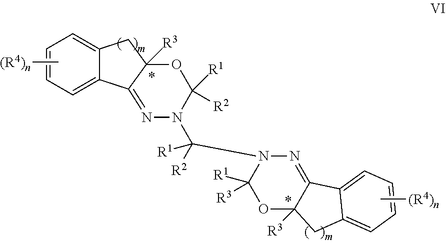
Wherein R¹, R²=hydrogen, C₁-C₄-alkyl
R³=C₁-C₄-alkoxycarbonyl, COOH
R⁴=Halogen, C₁-C₄alkyl, C₁-C₄ alkoxy
m=1-3
n=1-3
the process comprising the reaction of compound of formula V with compound of formula II 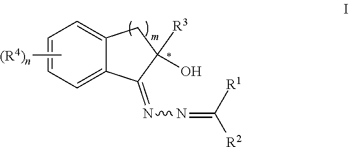
Optionally, in the presence of compound of formula III or IIIa 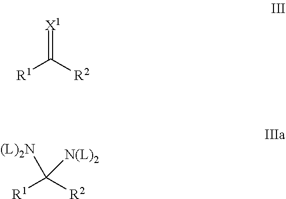
Wherein X¹=O, ⁺NL₂X⁻, L is hydrogen, C₁-C₄ alkyl, X is halogen, R¹, R² is independently hydrogen, C₁-C₄-alkyl,
in the presence of organic solvent and optionally, in the presence of catalyst.

23. A process of preparing of compound of formula VIa which is racemic or enantiomerically enriched at chiral center* 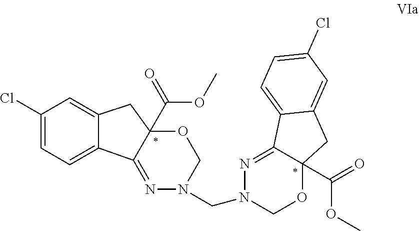
the process comprising the reaction of compound of formula IIa 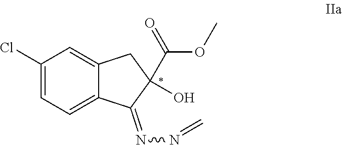
with dimethylmethyleneammonium chloride in the presence of organic solvent and optionally, in the presence of catalyst.

24. A process of preparing of compound of formula VIa which is racemic or enantiomerically enriched at chiral center* 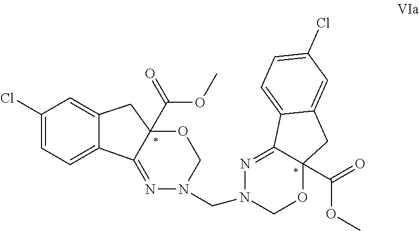
the process comprising the reaction of compound of formula IIa 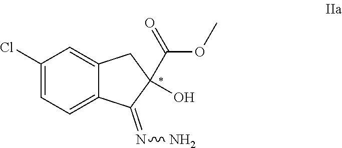
with tetramethylmethanediamine in the presence of organic solvent and optionally in the presence of catalyst.

25. A process of preparing of compound of formula VIa which is racemic or enantiomerically enriched at chiral center* 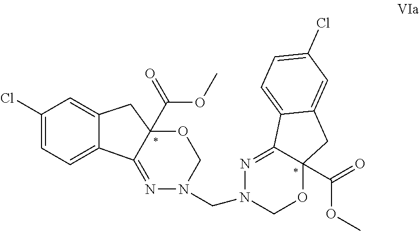
the process comprising the reaction of compound of formula Ia 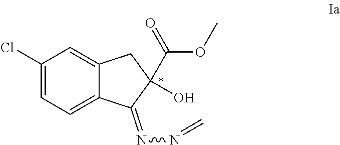
with formaldehyde optionally in the presence of organic solvent selected from the group consisting of toluene, xylene, monochlorobenzene, dichlorobenzene, ethylbenzene, cyclopentylmethyl ether, methyl-tert-butyl ether, benzonitrile and mixtures thereof.

26. The process according to claim 25 wherein the catalyst is present.

27. A process for preparing of compound of formula IV which is racemic or enantiomerically enriched at chiral center* 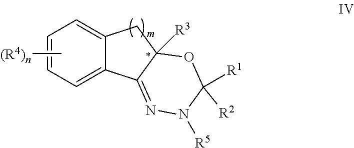
Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy 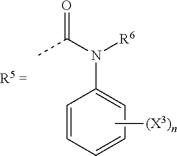
R⁶=hydrogen, CO₂Me, CO₂(R′)
X³=Halogen, OCF₃, SCF₃
n=1-3;
the process comprising the reaction of compound of formula I 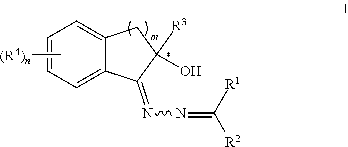
with a compound of formula VII: 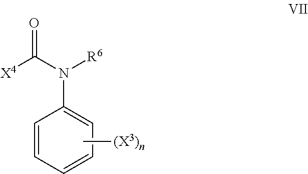
Wherein R⁶=hydrogen, CO₂Me, CO₂(R¹)
n=1-3
X³=Halogen, OCF₃, SCF₃
X⁴=Halogen, triflate, tosylate
in the presence of organic solvent.

28. The process according to claim 27, wherein the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

29. A process for preparation of compound of formula IVa which is racemic or enantiomerically enriched at chiral center* 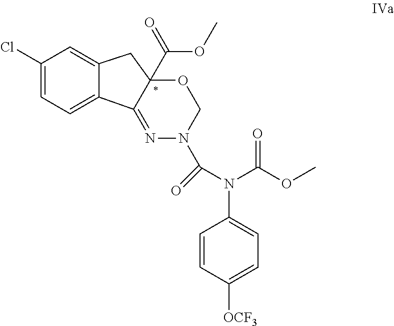
comprising the reaction of compound of formula Ia 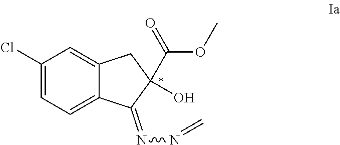
with a compound of formula VIIa 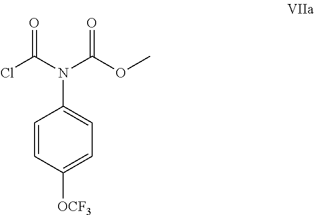 [SMILES: COC(=O)N(C(=O)Cl)c1ccc(OC(F)(F)F)cc1]
in the presence of organic solvent.

30. The process according to claim 29, wherein the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

31. A process for preparing of compound of formula IVa which is racemic or enantiomerically enriched at chiral center* 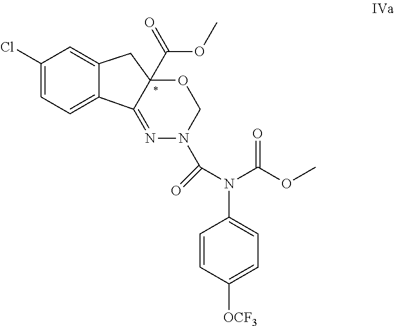
the process comprising the reaction of compound of formula VIa 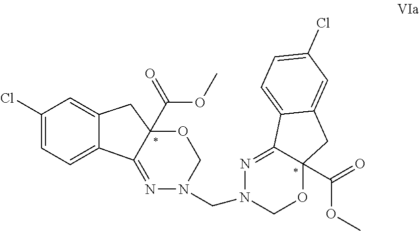
with a compound of formula VIIa 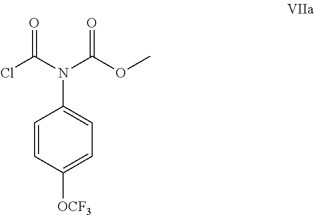 [SMILES: COC(=O)N(C(=O)Cl)c1ccc(OC(F)(F)F)cc1]
in the presence of organic solvent.

32. The process according to claim 31, wherein the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

33. A process for preparing of compound of formula IVa which is racemic or enantiomerically enriched at chiral center* 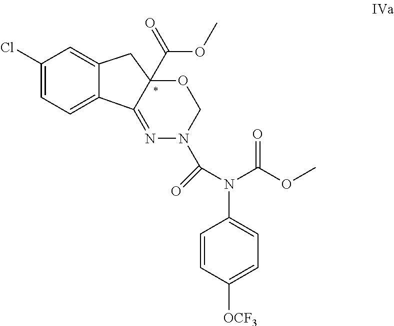
the process comprising the reaction of compound of formula Va 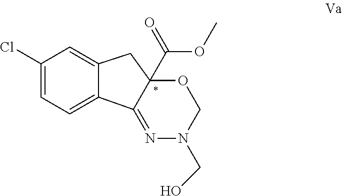
with a compound of formula VIIa 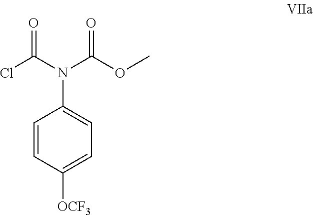 [SMILES: COC(=O)N(C(=O)Cl)c1ccc(OC(F)(F)F)cc1]
in the presence of organic solvent.

34. The process according to claim 33, wherein the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

35. A process for preparation of compound of formula IV which is racemic or enantiomerically enriched at chiral center* 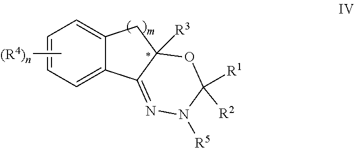
Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy 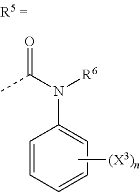
R⁶=hydrogen, CO₂Me, CO₂(R¹)
X³=Halogen, OCF₃, SCF₃
n=1-3;
comprising the reaction of at list one of the compounds of formula I, V and/or VI: 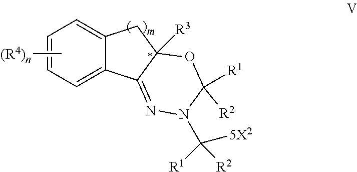
Wherein R¹, R² is independently hydrogen, C₁-C₄-alkyl
R³ is C₁-C₄-alkoxycarbonyl, COOH
R⁴ is halogen, C₁-C₄ alkyl, C₁-C₄alkoxy
X²=OR¹, N(R¹)₂
m=1-3
n=1-3 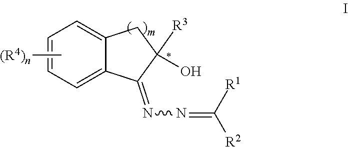
Wherein R¹, R² are independently hydrogen, C₁-C₄-alkyl
R³ is C₁-C₄-alkoxycarbonyl, COOH
R⁴ is halogen, C₁-C₄ alkyl, C₁-C₄ alkoxy
m=1-3
n=1-3 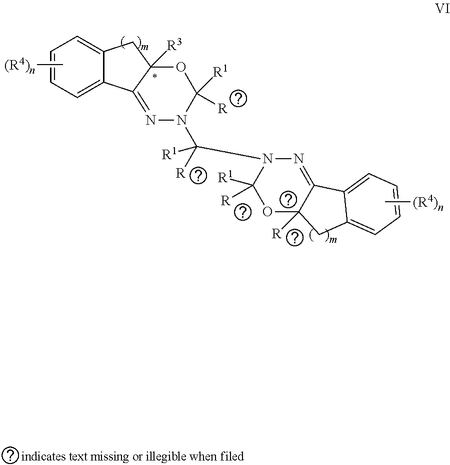
Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy
m=1-3
n=1-3
with a compound of formula VII in the presence of organic solvent. 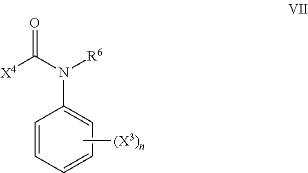
Wherein R⁶=hydrogen, CO₂Me, CO₂(R¹)
n=1-3
X³=Halogen, OCF₃, SCF₃
X⁴=Halogen, triflate, tosylate
in the presence of organic solvent.

36. A process for preparation of compound of formula IVa which is racemic or enantiomerically enriched at chiral center* 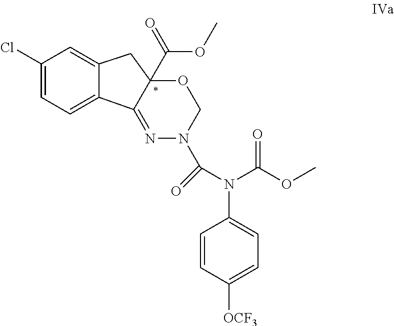
comprising the reaction of at list one of the compounds of formula Ia, Va and/or Via 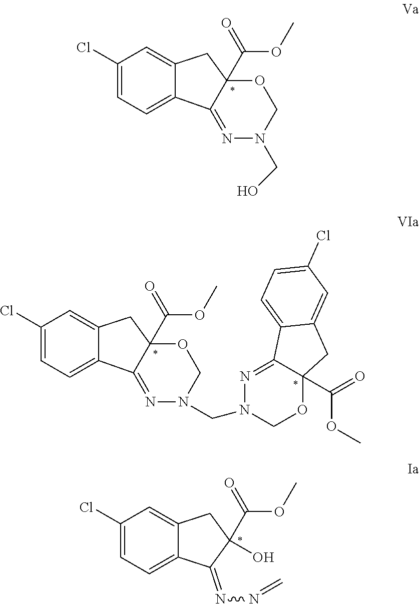
with a compound of formula Vila 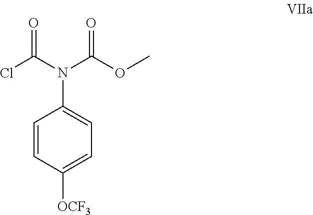 [SMILES: COC(=O)N(C(=O)Cl)c1ccc(OC(F)(F)F)cc1]
in the presence of organic solvent.

37. A process according to any of claims 35-36, wherein the organic solvent is aprotic solvent selected from the group consisting of toluene, xylene, monochlorobenzene, dichlorobenzene, ethylbenzene, cyclopentylmethyl ether, methyl-tert-butyl ether, benzonitrile and mixtures thereof.

38. A process according to claims 35-37 comprising the presence of water as co-solvent.

39. Purification of compound of formula IV prepared according to any of the preceding claims by recrystallization from C₁-C₄ alcohol, optionally in the presence of water.

40. Purification of compound of formula IV prepared according to any of the preceding claims by recrystallization from isopropanol, optionally in the presence of water.

41. A compound of formula VIII (methyl 8-chloro-2-((methoxycarbonyl)(4-(trifluoromethoxy)phenyl)carbamoyl)-2,6-dihydro-3H-indeno[1,2-e][1,3,4]oxadiazepine-5a(5H)-carboxylate which is racemic or enantiomerically enriched at chiral center*:

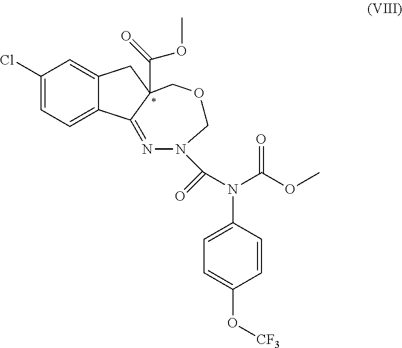 [SMILES: COC(=O)N(C(=O)N1COCC2(C(=O)OC)Cc3cc(Cl)ccc3C2=N1)c1ccc(OC(F)(F)F)cc1]

42. A process for preparing of compound of formula VIII which is racemic or enantiomerically enriched at chiral center* according to the following scheme, comprising: a) reacting the mixture of methyl (S)-5-chloro-2-hydroxy-1-oxo-2,3-dihydro-1H-indene-2-carboxylate and (1H-Indene-2-carboxylic acid, 5-chloro-2,3-dihydro-1-oxo-, methyl ester) with hydrazine hydrate in the presence of methanol and acetic acid, b) reacting the mixture of methyl (S)-5-chloro-1-hydrazineylidene-2-hydroxy-2,3-dihydro-1H-indene-2-carboxylate and methyl 5-chloro-1-hydrazineylidene-2,3-dihydro-1H-indene-2-carboxylate prepared in a) with formaldehyde in the presence of pyridine hydrochloride, c) reacting the mixture of methyl (S)-5-chloro-2-hydroxy-1-(methylenehydrazineylidene)-2,3-dihydro-1H-indene-2-carboxylate and methyl 8-chloro-2,6-dihydro-3H-indeno[1,2-e][1,3,4]oxadiazepine-5a(5H)-carboxylate, prepared in step b) with methyl N-carbonochloridoyl-N-[4-(trifluoromethoxy)phenyl]carbamate:

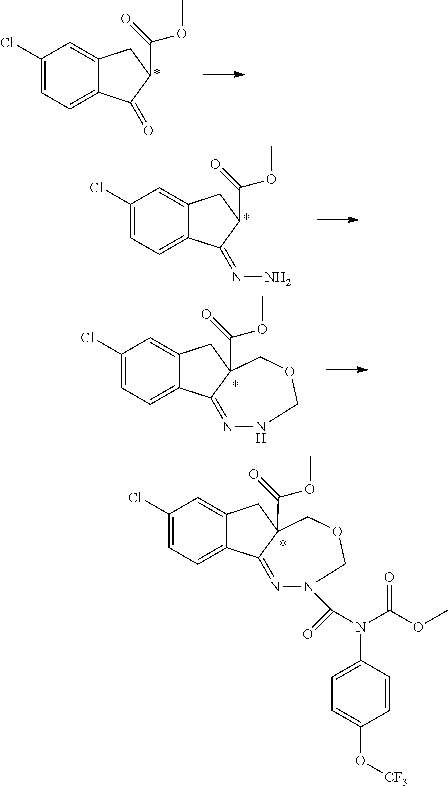

43. Purification of compound of formula VIII prepared according to claim 38 by recrystallization from the mixture of isopropanol/water, wherein the ratio of isopropanol to water is 3:1.

44. A telescopic process of for preparation of compound of formula IV which is racemic or enantiomerically enriched at chiral center* 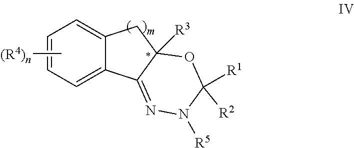
Wherein R¹, R²=hydrogen, C₁-C₄-alkyl
R³=C₁-C₄-alkoxycarbonyl, COOH
R⁴=Halogen, C₁-C₄ alkyl, C₁-C₄ alkoxy 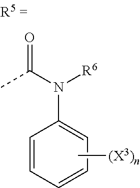
R⁶=hydrogen, CO₂Me, CO₂(R¹)
X³=Halogen, OCF₃, SCF₃
n=1-3;
comprising the steps of: a) preparation of compound I, or V, or VI or the mixture thereof; b) reaction of compound I, or V, or VI or the mixture thereof with compound of formula VII in the presence of organic solvent.

45. The process according to claim 44, wherein the organic solvent is aprotic solvent selected from the group comprising toluene, xylene, monochlorobenzene, dichlorobenzene, ethylbenzene, cyclopentylmethyl ether, methyl-tert-butyl ether, benzonitrile and mixtures thereof.
